(19) United States
(12) Patent Application Publication
(10) Pub. No.: US 2010/0016299 A1
(43) Pub. Date: Jan. 21, 2010

(54) Substituted benzoxepino-isoxazoles and use thereof

(75) Inventors: Nils Griebenow, Dormegen (DE); Anja Buchmüller, Essen (DE); Peter Kolkhof, Wuppertal (DE); Hilmar Bischoff, Wuppertal (DE)

Correspondence Address: EDWARDS ANGELL PALMER & DODGE LLP, P.O. BOX 55874, BOSTON, MA, 02205, US

(73) Assignee: BAYER HEALTHCARE AG, Leverkusen (DE)

(21) Appl. No.: 12/307,540

(22) PCT Filed: Jun. 28, 2007
(86) PCT No.: PCT/EP2007/005711

§ 371 Date: Jul. 7, 2009

(30) Foreign Application Priority Data
Jul. 6, 2006 (DE) 10 2006 031 176.0

(51) Int. Cl. A61K 31/424; C07D 498/04; A61K 31/454; A61K 31/5377; A61K 31/496; A61P 9/00

(52) U.S. Cl. 514/232.8; 548/242; 546/272.1; 544/137; 544/368; 514/379; 514/321; 514/254.04

(57) ABSTRACT

The present application relates to novel substituted benzoxepinoisoxazole derivatives, processes for their preparation, their use for the treatment and/or prophylaxis of diseases, and their use for producing medicaments for the treatment and/or prophylaxis of diseases, preferably for the treatment and/or prevention of cardiovascular disorders, especially of dyslipidemias, arteriosclerosis, restenosis and ischemias.

The present application relates to novel substituted benzoxepinoisoxazole derivatives, processes for their preparation, their use for the treatment and/or prophylaxis of diseases, and their use for producing medicaments for the treatment and/or prophylaxis of diseases, preferably for the treatment and/or prevention of cardiovascular disorders, especially of dyslipidemias, arteriosclerosis, restenosis and ischemias.

A large number of epidemiological studies has shown a causal connection between dyslipidemias and cardiovascular disorders. Elevated plasma cholesterol in isolation is one of the greatest risk factors for cardiovascular disorders such as, for example, arteriosclerosis. This relates both to an isolated hypercholesterolemia and to hypercholesterolemias combined with, for example, elevated plasma triglycerides or low plasma HDL-cholesterol. Substances which have a cholesterol- or combined cholesterol- and triglyceride-lowering effect ought therefore to be suitable for the treatment and prevention of cardiovascular disorders.

It has already been shown in animal models that plasma cholesterol and triglycerides are lowered by squalene synthase inhibitors. Squalene synthase (EC 2.5.1.21) catalyzes the conversion, by reductive condensation, of famesyl pyrophosphate into squalene. This is a crucial step in cholesterol biosynthesis. Whereas famesyl pyrophosphate and precursors are also of importance for other cellular metabolic pathways and reactions, squalene serves exclusively as precursor for cholesterol. Inhibition of squalene synthase thus leads directly to a reduction in cholesterol biosynthesis and thus to a fall in plasma cholesterol levels. It has additionally been shown that squalene synthase inhibitors also reduce plasma triglyceride levels. Inhibitors of squalene synthase might thus be employed for the treatment and/or prevention of cardiovascular disorders such as, for example, dyslipidemias, arteriosclerosis, ischemia/reperfusion, restenosis and arterial inflammations [cf., for example, Eur. Heart J. 19 (Suppl. A), A2-A11 (1998); Prog. Med. Chem. 33, 331-378 (1996); Europ. J. Pharm. 431, 345-352 (2001)].

It was an object of the present invention to provide novel compounds which can be employed as squalene synthase inhibitors for the treatment and/or prevention in particular of cardiovascular disorders.

WO 2005/068472 discloses certain tricyclic benzazepine derivatives as squalene synthase inhibitors. [2]Benzoxepino[4,5-c]isoxazole derivatives as such and the use thereof have not to date been described in the literature. This takes place for the first time in the context of the present invention.

The present invention relates to compounds of the general formula (I)

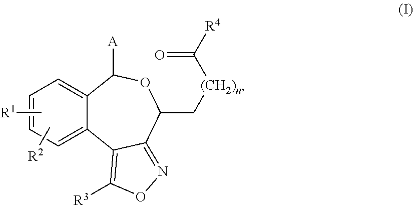

in which
A is (C₆-C₁₀)-aryl or 5- to 10-membered heteroaryl, which may each be substituted up to three times, identically or differently, by substituents selected from the group of halogen, cyano, nitro, trifluoromethyl, trifluoromethoxy, (C₁-C₆)-alkyl, (C₂-C₆)-alkenyl, (C₂-C₆)-alkynyl, (C₁-C₆)-alkoxy, hydroxy, amino, mono- and di-(C₁-C₆)-alkylamino,
   or
   is a group of the formula

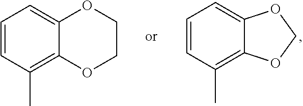

n is the number 0, 1, 2 or 3,
R¹ and R² are identical or different and are independently of one another, hydrogen, halogen, cyano, nitro, trifluoromethyl, trifluoromethoxy, (C₁-C₆)-alkyl or (C₁-C₆)-alkoxy,
R³ is (C₁-C₈)-alkyl, (C₂-C₈)-alkenyl, (C₂-C₈)-alkynyl, each of which may be substituted by (C₃-C₈)-cycloalkyl, or is (C₃-C₈)-cycloalkyl, where
   (C₁-C₈)-alkyl, (C₂-C₈)-alkenyl, (C₂-C₈)-alkynyl and (C₃-C₈)-cycloalkyl may each be substituted by fluorine, hydroxy, amino, (C₁-C₄)-alkoxy or (C₁-C₄)-acyloxy,
and
R⁴ is a group of the formula —OR⁵ or —NR⁶R⁷ in which
   R⁵ is hydrogen or (C₁-C₆)-alkyl,
   R⁶ and R⁷ are identical or different and are independently of one another hydrogen, (C₁-C₆)-alkyl or (C₃-C₈)-cycloalkyl, each of which may be substituted by substituents selected from the group of carboxyl, (C₁-C₆)-alkoxycarbonyl, aminocarbonyl, mono- and di-(C₁-C₆)-alkylaminocarbonyl,
   or
   R⁶ and R⁷ together with the nitrogen atom to which they are bonded form a 4- to 8-membered heterocycle which may comprise a further ring heteroatom from the series N—R⁸, O, S, SO or SO₂ and be substituted by substituents selected from the group of hydroxy, oxo, amino, (C₁-C₆)-alkyl, carboxyl, (C₁-C₆)-alkoxycarbonyl, aminocarbonyl, mono- and di-(C₁-C₆)-alkylaminocarbonyl, in which
      (C₁-C₆)-alkyl may in turn be substituted by substituents selected from the group of fluorine, hydroxy, amino, carboxyl, (C₁-C₆)-alkoxycarbonyl, aminocarbonyl, mono- and di-(C₁-C₆)-alkyaminocarbonyl,
      and
      R⁸ is hydrogen, (C₁-C₄)-alkyl, (C₁-C₄)-acyl or (C₁-C₄)-alkoxycarbonyl,
and the salts, solvates and solvates of the salts thereof.

Compounds according to the invention are the compounds of the formula (I) and the salts, solvates and solvates of the salts thereof, the compounds which are encompassed by formula (I) and are of the formulae mentioned hereinafter, and the salts, solvates and solvates of the salts thereof, and the compounds which are encompassed by formula (I) and are mentioned hereinafter as exemplary embodiments, and the salts, solvates and solvates of the salts thereof, insofar as the compounds encompassed by formula (I) and mentioned hereinafter are not already salts, solvates and solvates of the salts.

The compounds of the invention may, depending on their structure, exist in stereoisomeric forms (enantiomers, diastereomers). The invention therefore encompasses the enantiomers or diastereomers and respective mixtures thereof The stereoisomerically pure constituents can be isolated in a known manner from such mixtures of enantiomers and/or diastereomers.

Where the compounds of the invention can occur in tautomeric forms, the present invention encompasses all tautomeric forms.

Salts preferred for the purposes of the present invention are physiologically acceptable salts of the compounds of the invention. However, salts which are themselves unsuitable for pharmaceutical applications but can be used for example for isolating or purifying the compounds of the invention are also encompassed.

Physiologically acceptable salts of the compounds of the invention include acid addition salts of mineral acids, carboxylic acids and sulfonic acids, e.g. salts of hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, acetic acid, trifluoroacetic acid, propionic acid, lactic acid, tartaric acid, malic acid, citric acid, fumaric acid, maleic acid and benzoic acid.

Physiologically acceptable salts of the compounds of the invention also include salts of conventional bases such as, for example and preferably, alkali metal salts (e.g. sodium and potassium salts), alkaline earth metal salts (e.g. calcium and magnesium salts) and ammonium salts derived from ammonia or organic amines having 1 to 16 C atoms, such as, for example and preferably, ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine and N-methylpiperidine.

Solvates refer for the purposes of the invention to those forms of the compounds of the invention which form a complex in the solid or liquid state through coordination with solvent molecules. Hydrates are a specific form of solvates in which the coordination takes place with water. Solvates preferred in the context of the present invention are hydrates.

The present invention also encompasses prodrugs of the compounds according to the invention. The term “prodrugs” encompasses compounds which themselves may be biologically active or inactive but are converted during their residence time in the body into compounds according to the invention (for example by metabolism or hydrolysis).

In the context of the present invention, the substituents have the following meaning unless otherwise specified:

(C₁-C₈)-Alkyl, (C₁-C₆)-alkyl and (C₁-C₄)-alkyl are in the context of the invention a straight-chain or branched alkyl radical having respectively 1 to 8, 1 to 6 and 1 to 4 carbon atoms. A straight-chain or branched alkyl radical having 1 to 6 or 1 to 4 carbon atoms is preferred. A straight-chain or branched alkyl radical having 1 to 4 carbon atoms is particularly preferred. Examples which may be preferably mentioned are: methyl, ethyl, n-propyl, isopropyl, n-butyl, iso-butyl, sec-butyl, tert-butyl, 1 -ethylpropyl, n-pentyl and n-hexyl.

(C₂-C₈)-Alkenyl and (C₂-C₆)-alkenyl in the context of the invention are a straight-chain or branched alkenyl radical having respectively 2 to 8 and 2 to 6 carbon atoms and one or two double bonds. A straight-chain or branched alkenyl radical having 2 to 6 carbon atoms is preferred, particularly preferably having 2 to 4 carbon atoms and one double bond. Examples which may be preferably mentioned are: vinyl, allyl, isopropenyl and n-but-2-en-1-yl.

(C₂-C₈)-Alkynyl and (C₂-C₆)-alkynyl in the context of the invention are a straight-chain or branched alkynyl radical having respectively 2 to 8 and 2 to 6 carbon atoms and a triple bond. A straight-chain or branched alkynyl radical having 2 to 6 carbon atoms is preferred, particularly preferably having 2 to 4 carbon atoms. Examples which may be preferably mentioned are: ethynyl, n-prop-1-yn-1-yl, n-prop-2-yn-1-yl, n-but-2-yn-1-yl and n-but-3-yn-1-yl.

(C₃-C₈)-Cycloalkyl and (C₃-C₆)-cycloalkyl in the context of the invention are a monocyclic, saturated cycloalkyl group having respectively 3 to 8 and 3 to 6 carbon atoms. A cycloalkyl radical having 3 to 6 carbon atoms is preferred. Examples which may be preferably mentioned are: cyclopropyl, cyclobutyl, cyclopentyl, cyclohexyl and cycloheptyl.

(C₆-C₁₀)-Aryl in the context of the invention is an aromatic radical having preferably 6 to 10 carbon atoms. Preferred aryl radicals are phenyl and naphthyl.

(C₁-C₆)-Alkoxy and (C₁-C₄)-alkoxy in the context of the invention are a straight-chain or branched alkoxy radical having respectively 1 to 6 and 1 to 4 carbon atoms. A straight-chain or branched alkoxy radical having 1 to 4 carbon atoms is preferred. Examples which may be preferably mentioned are: methoxy, ethoxy, n-propoxy, isopropoxy and tert-butoxy.

(C₁-C₆)-Alkoxycarbonyl and (C₁-C₄)-alkoxycarbonyl in the context of the invention are a straight-chain or branched alkoxy radical having respectively 1 to 6 and 1 to 4 carbon atoms which is linked via a carbonyl group. A straight-chain or branched alkoxycarbonyl radical having 1 to 4 carbon atoms in the alkoxy group is preferred. Examples which may be preferably mentioned are: methoxy-carbonyl, ethoxycarbonyl, n-propoxycarbonyl, isopropoxycarbonyl and tert-butoxycarbonyl.

Mono-(C₁-C₆)-alkylamino and mono-(C₁-C₄)-alkylamino in the context of the invention are an amino group having a straight-chain or branched alkyl substituent which has respectively 1 to 6 and 1 to 4 carbon atoms. A straight-chain or branched monoalkylamino radical having 1 to 4 carbon atoms is preferred. Examples which may be preferably mentioned are: methylamino, ethylamino, n-propylamino, isopropylamino and tert-butylamino.

Di-(C₁-C₆)-alkylamino and di-(C₁-C₄)-alkylamino in the context of the invention are an amino group having two identical or different straight-chain or branched alkyl substituents which each have respectively 1 to 6 and 1 to 4 carbon atoms. Straight-chain or branched dialkylamino radicals having in each case 1 to 4 carbon atoms are preferred. Examples which may be preferably mentioned are: N,N-dimethylamino, N,N-diethylamino, N-ethyl-N-methylamino, N-methyl-N-n-propylamino, N-isopropyl-N-n-propylamino, N-tert-butyl-N-methylamino, N-ethyl-N-n-pentylamino and N-n-hexyl-N-methylamino.

Mono- or di-(C₁-C₆)-alkylaminocarbonyl and mono- or di-(C₁-C₄)-alkylaminocarbonyl in the context of the invention are an amino group which is linked via a carbonyl group and which has respectively a straight-chain or branched and two identical or different straight-chain or branched alkyl substituents each having respectively 1 to 6 and 1 to 4 carbon atoms. Examples which may be preferably mentioned are: methylaminocarbonyl, ethylaminocarbonyl, isopropylaminocarbonyl, tert-butylaminocarbonyl, N,N-dimethylaminocarbonyl, N,N-diethylaminocarbonyl, N-ethyl-N-methylaminocarbonyl and N-tert-butyl-N-methylaminocarbonyl.

(C₁-C₄)-Acyl [(C₁-C₄)-alkanoyl] in the context of the invention is a straight-chain or branched alkyl radical having 1 to 4 carbon atoms which has a doubly bonded oxygen atom in position 1 and is linked via position 1. Examples which may be preferably mentioned are: formyl, acetyl, propionyl, n-butyryl and iso-butyryl.

(C₁-C₄)-Acyloxy in the context of the invention is a straight-chain or branched alkyl radical having 1 to 4 carbon atoms which has a doubly bonded oxygen atom in position 1 and is linked via a further oxygen atom in position 1. Examples which may be preferably mentioned are: acetoxy, propionoxy, n-butyroxy and iso-butyroxy.

5- to 10-membered heteroaryl in the context of the invention is a mono- or, where appropriate, bicyclic aromatic heterocycle (heteroaromatic system) having up to three identical or different heteroatoms from the series N, O and/or S, which is linked via a ring carbon atom or, where appropriate, via a ring nitrogen atom of the heteroaromatic system. Examples which may be mentioned are: furanyl, pyrrolyl, thienyl, pyrazolyl, imidazolyl, thiazolyl, oxazolyl, isoxazolyl, iso-thiazolyl, pyridyl, pyrimidinyl, pyridazinyl, pyrazinyl, benzofuranyl, benzothienyl, benzimidazolyl, benzoxazolyl, indolyl, indazolyl, quinolinyl, isoquinolinyl, naphthyridinyl, quinazolinyl, quinoxalinyl. 5- to 6-membered heteroaryl radicals having up to two heteroatoms from the series N, O and/or S are preferred, such as, for example, furyl, thienyl, thiazolyl, oxazolyl, isothiazolyl, isoxazolyl, imidazolyl, pyridyl, pyrimidinyl, pyridazinyl, pyrazinyl.

A 4- to 8-, 5- to 7- or 5- to 6-membered heterocycle in the context of the invention is a saturated or partially unsaturated heterocycle having in total respectively 4 to 8, 5 to 7 and 5 to 6 ring atoms which comprises a ring nitrogen atom, is linked via the latter and may comprise a further heteroatom from the series N, O, S, SO or SO₂. A 5- to 7-membered saturated, N-linked heterocycle which may comprise a further heteroatom from the series N, O or S is preferred. Examples which may be mentioned are: pyrrolidinyl, pyrrolinyl, piperidinyl, piperazinyl, morpholinyl, thiomorpholinyl, azepinyl, 1,4-diazepinyl. Piperidinyl, piperazinyl, morpholinyl and pyrrolidinyl are particularly preferred.

Halogen in the context of the invention includes fluorine, chlorine, bromine and iodine. Chlorine or fluorine are preferred.

If radicals in the compounds according to the invention are substituted, the radicals may, unless otherwise specified, be substituted one or more times. In the context of the present invention, all radicals which occur more than once have a mutually independent meaning. Substitution by one, two or three identical or different substituents is preferred. Substitution by one substituent is very particularly preferred.

Preference is given to compounds of the formula (I) in which
A is phenyl, naphthyl or pyridyl, each of which may be substituted up to twice, identically or differently, by substituents selected from the group of fluorine, chlorine, bromine, cyano, nitro, trifluoromethyl, trifluoromethoxy, (C₁-C₄)-alkyl, (C₁-C₄)-alkoxy, hydroxy, amino, mono- and di-(C₁-C₄)-alkylamino,
n is the number 0 or 1,
R¹ and R² are identical or different and are independently of one another, hydrogen, fluorine, chlorine, bromine, cyano, nitro, trifluoromethyl, trifluoromethoxy, (C₁-C₄)-alkyl or (C₁-C₄)-alkoxy,
R³ is (C₁-C₆)-alkyl which may be substituted by (C₃-C₆)-cycloalkyl, or is (C₃-C₆)-cycloalkyl, where
   (C₁-C₆)-alkyl and (C₃-C₆)-cycloalkyl may each be substituted by hydroxy, amino, (C₁-C₄)-alkoxy or (C₁-C₄)-acyloxy,
and
R⁴ is a group of the formula —OR⁵ or —NR⁶R⁷ in which
   R⁵ is hydrogen or (C₁-C₆)-alkyl,
R⁶ and R⁷ are identical or different and are independently of one another hydrogen, (C₁-C₆)-alkyl or (C₃-C₆)-cycloalkyl, each of which may be substituted by substituents selected from the group of carboxyl, (C₁-C₆)-alkoxycarbonyl, aminocarbonyl, mono- and di-(C₁-C₆)-alkylaminocarbonyl,
   or
   R⁶ and R⁷ together with the nitrogen atom to which they are bonded form a 5- to 7-membered heterocycle which may comprise a further ring heteroatom from the series N—R⁸, O or S and be substituted by substituents selected from the group of hydroxy, oxo, amino, (C₁-C₆)-alkyl, carboxyl, (C₁-C₆)-alkoxycarbonyl, amino-carbonyl, mono- and di-(C₁-C₆)-alkylaminocarbonyl, in which
      (C₁-C₆)-alkyl in turn may be substituted by substituents selected from the group of hydroxy, amino, carboxyl, (C₁-C₆)-alkoxycarbonyl, aminocarbonyl, mono- and di-(C₁-C₆)-alkylaminocarbonyl,
      and
      R⁸ is hydrogen, (C₁-C₄)-alkyl, (C₁-C₄)-acyl or (C₁-C₄)-alkoxycarbonyl,
and the salts, solvates and solvates of the salts thereof

Particular preference is given to compounds of the formula (I) in which
A is phenyl which is substituted once or twice, identically or differently, by fluorine, chlorine, bromine, methyl, methoxy, ethoxy or dimethylamino,
n is the number 0,
R¹ and R² are independently of one another hydrogen or chlorine,
R³ is (C₁-C₆)-alkyl which may be substituted by (C₃-C₆)-cycloalkyl, or is (C₃-C₆)-cycloalkyl, where
   (C₁-C₆)-alkyl and (C₃-C₆)-cycloalkyl may each be substituted by hydroxy or (C₁-C₄)-acyloxy,
and
R⁴ is a group of the formula —OR⁵ or —NR⁶R⁷ in which
   R⁵ is hydrogen or (C₁-C₄)-alkyl,
      R⁶ and R⁷ are identical or different and are independently of one another hydrogen or (C₁-C₄)-alkyl which may be substituted by carboxyl or (C₁-C₄)-alkoxycarbonyl,
   or
   R⁶ and R⁷ together with the nitrogen atom to which they are bonded form a 5- or 6-membered heterocycle which may comprise a further ring heteroatom from the series N—R⁸ and O and be substituted by substituents selected from the group of hydroxy, oxo, amino, (C₁-C₄)-alkyl, carboxyl, (C₁-C₄)-alkoxycarbonyl, amino-carbonyl, mono- and di-(C₁-C₄)-alkylaminocarbonyl, in which
      (C₁-C₄)-alkyl in turn may be substituted by substituents selected from the group of hydroxy, amino, carboxyl, (C₁-C₄)-alkoxycarbonyl, aminocarbonyl, mono- and di-(C₁-C₄)-alkylaminocarbonyl,
      and
      R⁸ is hydrogen, (C₁-C₄)-alkyl or (C₁-C₄)-acyl,
and the salts, solvates and solvates of the salts thereof

Very particular preference is given to compounds of the formula (I) in which
A is phenyl which is substituted once or twice, identically or differently, by fluorine, chlorine, methyl, methoxy or ethoxy,
n is the number 0,
R¹ and R² are independently of one another hydrogen or chlorine,
R³ is (C₁-C₆)-alkyl,
and
R⁴ is hydroxy or a group of the formula —NR⁶R⁷ in which
   R⁶ and R⁷ together with the nitrogen atom to which they are bonded form a 5- or 6-membered heterocycle which may comprise a further ring heteroatom from the series N—R⁸ and O and be substituted by hydroxy, oxo, (C₁-C₄)-alkyl, carboxyl or (C₁-C₄)-alkoxycarbonyl, in which
      (C₁-C₄)-alkyl in turn may be substituted by hydroxy, carboxyl or (C₁-C₄)-alkoxycarbonyl,
      and
      R⁸ is hydrogen, methyl or acetyl,
and the salts, solvates and solvates of the salts thereof.

The definitions of radicals indicated specifically in the respective combinations or preferred combinations of radicals are replaced as desired irrespective of the particular combinations indicated for the radicals also by the definitions of radicals of other combinations.

Combinations of two or more of the abovementioned preferred ranges are very particularly preferred.

The invention further relates to a process for preparing the compounds of the invention, characterized in that a compound of the formula (II)

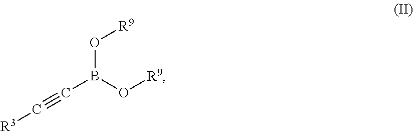

in which R³ has the meaning indicated above, and
R⁹ is hydrogen, (C₁-C₆)-alkyl or (C₃-C₆)-cycloalkyl, or the two R⁹ radicals together form an ortho-phenylene, —CH₂—CH₂—, —C(CH₃)₂—C(CH₃)₂—, —CH₂—C(CH₃)₂—CH₂— or —CH₂—CHR¹⁰—CH₂ bridge in which
   R¹⁰ is hydrogen, (C₁-C₄)-alkyl or (C₁-C₄)-alkoxy,
is reacted in an inert solvent under basic conditions with a compound of the formula (III)

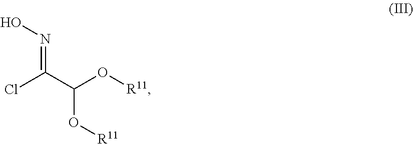

in which
R¹¹ is (C₁-C₆)-alkyl or (C₃-C₆)-cycloalkyl, or the two R¹¹ radicals together form an ortho-phenyl —CH₂—CH₂—, —C(CH₃)₂—C(CH₃)₂—, —CH₂—C(CH₃)₂—CH₂— or —CH₂—CHR¹²—CH₂— bridge in which
   R¹² is hydrogen, (C₁-C₄)-alkyl or (C₁-C₄)-alkoxy,
to give a compound of the formula (IV)

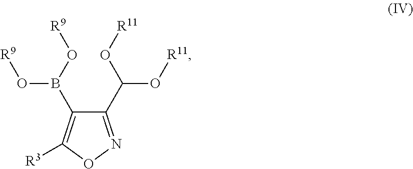

in which R³, R⁹ and R¹¹ each have the meanings indicated above,
   [cf., for example, Davies et al., Chem. Commun., 1558-1559 (2001)], the latter is coupled in an inert solvent in the presence of a transition metal catalyst and of a base with a compound of the formula (V)

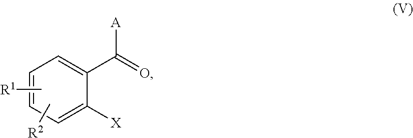

in which R¹, R² and A each have the meanings indicated above, and
X is halogen, in particular chlorine, bromine or iodine, or is a halogen equivalent by way of example and preferably from the group of alkylsulfonates or arylsulfonates such as, for example, mesylate, triflate, tresylate, nonaflate or tosylate,
to give a 4-phenylisoxazole derivative of the formula (VI)

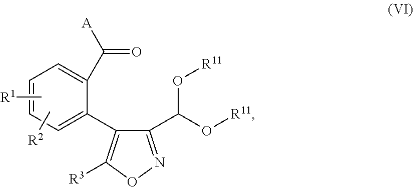

in which R¹, R², R³, R¹¹ and A each have the meanings indicated above,
   [cf., for example, Moore et al., Synlett, 2071-2073 (2002)], subsequently converted under acidic conditions into an aldehyde of the formula (VII)

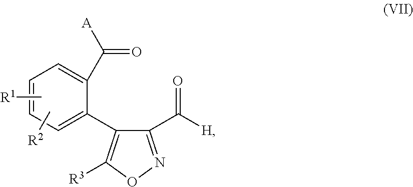

in which R¹, R², R³ and A each have the meanings indicated above,
   [cf., for example, Greene et al., Protective Groups in Organic Synthesis, 2^{nd} edition, Wiley, New York, 1991, pages 178-183], subsequently reacted in an inert solvent by a Wittig reaction with phosphorus ylides or their tautomeric phosphorus ylenes such as, for example, ethoxycarbonyl-methylenetriphenylphosphorane, or by Wittig-Homer reaction with so-called PO ylides to give a compound of the formula (VIII)

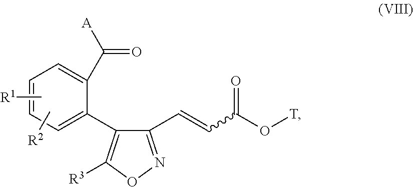

in which R¹, R², R³ and A each have the meanings indicated above, and
T is (C₁-C₄)-alkyl,
   [cf., for example, Wittig et al., Justus Liebigs Ann. Chem. 508, 44-57 (1953); Maryanoff et al., Chem. Rev. 89, 863-927 (1989); Schlosser, Chemie für Labor und Betrieb 33 (6), 259-263 (1982) then reduced in an inert solvent with the aid of a boron hydride or aluminum hydride, such as, for example, sodium borohydride, potassium borohydride or lithium tri(tert-butyloxy)aluminum hydride, to a compound of the formula (IX)

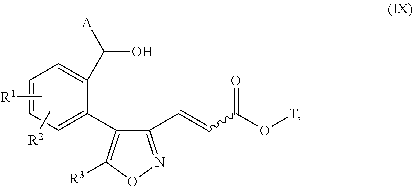

in which R¹, R², R³, A and T each have the meanings indicated above,
   [cf., for example, Miki et al., Bioorg. Med. Chem. 10, 401-414 (2002)], the latter is subsequently cyclized in an inert solvent in the presence of a base to give a compound of the formula (I-A)

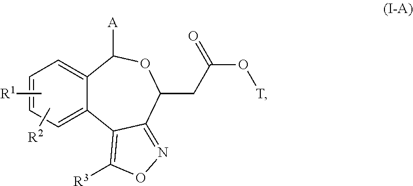

in which R¹, R², R³, A and T each have the meanings indicated above,
   [cf., for example, Miki et al., J. Med. Chem. 45 (20), 4571-4580 (2002)], then hydrolyzed under acidic conditions to give a carboxylic acid of the formula (I-B)

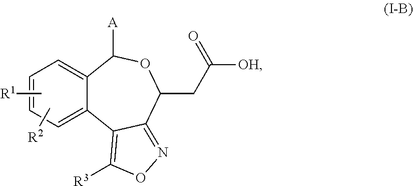

in which R¹, R², R³ and A each have the meanings indicated above,
   the compounds of the formula (I-B) are where appropriate converted by methods known from the literature for chain extension, such as, for example, by the Arndt-Eistert reaction (C₁ chain extension) [Eistert et al., Ber. Dtsch. Chem. Ges. 60, 1364-1370 (1927); Ye et al., Chem. Rev. 94, 1091-1160 (1994); Cesar et al., Tetrahedron Lett. 42, 7099-7102 (2001)], derivatization with Meldru's acid [cf. Smrcina, Tetrahedron 53, 12867-12874 (1997)] or reaction with N-hydroxy-2-thiopyridone [cf. Barton et al., Tetrahedron Lett. 32, 3309-3312 (1991)] (C₂ chain extension), or by the method of Steglich [cf. Steglich et al., Angew. Chem. 10, 655-656 (1971)] (C₃ chain extension), into the homologous carboxylic acids of the formula (I-C)

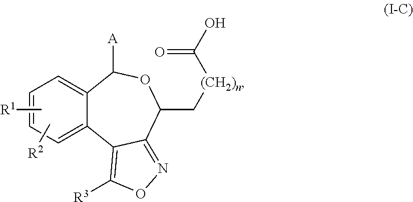

in which R¹, R², R³, A and n each have the meanings indicated above, but where n is not the number 0,
   and the resulting compounds of the formula (I-B) or (I-C) are then reacted by methods known from the literature for esterification or amidation of carboxylic acids with a compound of the formula (X) or (XI)

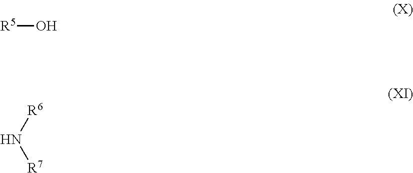

in which R⁵, R⁶ and R⁷ each have the meanings indicated above, but where R⁵ is not hydrogen,
to give the compounds of the formula (I),
and the compounds of the formula (I) are converted where appropriate with the appropriate (i) solvents and/or (ii) bases or acids into the solvates, salts and/or solvates of the salts thereof.

Separation of the compounds of the invention into the corresponding enantiomers and/or diastereomers is possible, as expedient, at the stage of the compounds (I-A), (I-B), (I-C) or (I); such a fractionation of the stereoisomers can be carried out by methods known to the skilled worker, preferably by chromatographic means.

Inert solvents for process step (II)+(III)→(IV) are for example ethers such as diethyl ether, tert-butyl methyl ether, dioxane, tetrahydrofuran, glycol dimethyl ether or diethylene glycol dimethyl ether, hydrocarbons such as benzene, toluene, xylene, hexane, cyclohexane or petroleum fractions, halohydrocarbons such as dichloromethane, trichloromethane, tetrachloromethane, 1,2-dichloroethane, trichloroethylene or chlorobenzene, or other solvents such as, for example, ethyl acetate, dimethylformamide or acetonitrile. It is likewise possible to employ mixtures of the solvents mentioned. Diethyl ether and glycol dimethyl ether (1,2-dimethoxyethane) are preferred.

Suitable bases are the usual inorganic or organic bases. These include in particular alkali metal bicarbonates such as sodium or potassium bicarbonate or amines such as, for example, triethylamine. Potassium bicarbonate is preferred.

The compound of the formula (III) is in this case employed in an amount of from 0.5 to 5 mol, preferably from 1 to 1.5 mol, based on 1 mol of the compound of the formula (II). The reaction generally takes place in a temperature range from +20° C. to +150° C., preferably at +50° C. to +80° C. The reaction can be carried out under atmospheric, elevated or reduced pressure (e.g. from 0.5 to 5 bar). It is generally carried out under atmospheric pressure.

The reaction (IV)+(V)→(VI) [“Suzuki reaction”; cf., for example, Suzuki et al., Synlett 3, 207-210 (1992); Suzuki et al., Chem. Rev. 95, 2457-2483 (1995)] takes place in the presence of a transition metal catalyst, such as palladium or nickel catalysts, and of a base.

Suitable solvents for this reaction are inert organic solvents which are not changed under the reaction conditions. These include for example ethers such as diethyl ether, tert-butyl methyl ether, dioxane, tetrahydrofuran, glycol dimethyl ether or diethylene glycol dimethyl ether, hydrocarbons such as benzene, toluene, xylene, hexane, cyclohexane or petroleum fractions, or other solvents such as, for example, ethyl acetate, dimethylformamide or acetonitrile. It is likewise possible to employ mixtures of the solvents mentioned. Dioxane is preferably used.

Suitable as base for the reaction (IV)+(V)→(VI) are customary inorganic or organic bases. These include in particular alkali metal carbonates such as sodium, potassium or cesium carbonate, alkali metal hydroxides such as lithium, sodium or potassium hydroxide, alkaline earth metal hydroxides such as bariuim hydroxide, alkali metal fluorides such as sodium, potassium or cesium fluoride, alkali metal alcoholates such as sodium ethanolate, alkali metal phosphates such as potassium phosphate, or organic amines such as, for example, triethylamine. Potassium phosphate is preferred.

Suitable as transition metal catalyst are for example [1,1′-bis(diphenylphosphino)ferrocene]di-chloropalladium(II), bis(triphenylphosphine)palladium(II) chloride or tetrakis(triphenyl-phosphine)palladium(0), or mixtures of transition metal complexes with complex ligands such as, for example, bis(dibenzylideneacetone)palladium(0)/bis(diphenylphosphino)ferrocene or bis(di-benzylideneacetone)palladium(0)/tri-tert-butylphosphine, or mixtures of transition metal salts with complex ligands such as, for example, palladium(II) acetate/tri-ortho-tolylphosphine. [1,1′-Bis(diphenylphosphino)ferrocene]dichloropalladium(II) is preferred. The catalyst is employed in this case in an amount of from 0.001 to 1 mol, preferably from 0.01 to 0.2 mol, based on 1 mol of the compound of the formula (IV).

The compound of the formula (IV) is employed in an amount of from 0.5 to 5 mol, preferably from 1 to 2.5 mol, based on 1 mol of the compound of the formula (V). The reaction generally takes place in a temperature range from +20° C. to +150° C., preferably at +60° C. to +100° C. The re carried out under atmospheric, elevated or reduced pressure (e.g. from 0.5 to 5 bar). It is generally carried out under atmospheric pressure.

Inert solvents for process step (VI)→(VII) are for example ethers such as diethyl ether, tert-butyl methyl ether, dioxane, tetrahydrofuran, glycol dimethyl ether or diethylene glycol dimethyl ether, alcohols such as methanol, ethanol, n-propanol, isopropanol, n-butanol or tert-butanol, or dipolar aprotic solvents such as acetone, dimethylformamide, dimethyl sulfoxide or acetonitrile, or else water. It is likewise possible to employ mixtures of the solvents mentioned. Tetrahydrofuran/water mixtures are preferred.

Suitable acids for process step (VI)→(VII) are aqueous solutions of the usual inorganic acids such as, for example, hydrochloric acid, sulfuric acid, phosphoric acid or hydrobromic acid. It is also possible to employ organic acids such as formic acid, trifluoroacetic acid, trifluoromethanesulfonic acid or p-toluenesulfonic acid, in each case with addition of water. Also suitable are acidic ion exchanger resins such as, for example, Amberlyst 15®, Dowex 50WX8®, Amberlite IR-120® or Purolite CT269®. Hydrochloric acid is preferably used.

The reaction generally takes place in a temperature range from +20° C. to +150° C., preferably at +50° C. to +I00° C. The reaction can be carried out under atmospheric, elevated or reduced pressure (e.g. from 0.5 to 5 bar). It is generally carried out under atmospheric pressure.

Inert solvents for process step (VII)→(VIII) are for example ethers such as diethyl ether, tert-butyl methyl ether, dioxane, tetrahydrofuran, glycol dimethyl ether or diethylene glycol dimethyl ether, hydrocarbons such as benzene, toluene, xylene, hexane, cyclohexane or petroleum fractions, halohydrocarbons such as dichloromethane, trichloromethane or chlorobenzene, or other solvents such as, for example, ethyl acetate or acetonitrile. It is likewise possible to employ mixtures of the solvents mentioned. Tetrahydrofuran, dichloromethane or toluene are preferably used.

Suitable phosphorus ylides or phosphorus ylenes for the Wittig reaction are for example ethoxy-carbonylmethylenetriphenylphosphorane or tert-butoxycarbonylmethylenetriphenylphosphorane. These phosphorus ylides or ylenes can also be obtained from the corresponding phosphonium salts such as, for example, ethoxycarbonylmethyltriphenylphosphonium bromide via the action of a base such as, for example, sodium hydride, potassium tert-butanolate or 1,5,7-triazabicyclo[4.4.0]dec-5-ene. It is also possible to employ in a Wittig-Horner reaction so-called PO ylides which can be obtained from the appropriate phosphonic esters such as, for example, triethylphosphonoacetate in the presence of a base such as, for example, sodium hydride or 1,5,7-triazabicyclo[4.4.0]dec-5-ene.

The ylides or ylenes described above are in this case employed in an amount of from 0.5 to 5 mol, preferably from 1 to 1.5 mol, based on 1 mol of the compound of the formula (VII). The reaction generally takes place in a temperature range from −40° C. to +100° C., preferably at 0° C. to +40° C. The reaction can be carried out under atmospheric, elevated or reduced pressure (e.g. from 0.5 to 5 bar). It is generally carried out under atmospheric pressure.

Inert solvents for process step (VIII)→(IX) are for example ethers such as diethyl ether, tert-butyl methyl ether, dioxane, tetrahydrofuran, glycol dimethyl ether or diethylene glycol dimethyl ether, alcohols such as methanol, ethanol, n-propanol, isopropanol, n-butanol or tert-butanol, or hydrocarbons such as benzene, toluene, xylene, hexane, cyclohexane or petroleum fractions, or other solvents such as, for example, ethyl acetate. It is likewise possible to employ mixtures of the solvents mentioned. Tetrahydrofuran is preferably used.

Suitable reducing agents are boron hydrides or aluminum hydrides such as, for example, lithium borohydride, sodium borohydride, potassium borohydride or lithium tri(tert-butyloxy)aluminum hydride. Lithium tri(tert-butyloxy)aluminum hydride is preferably used.

The reaction generally takes place in a temperature range from −40° C. to +100° C., preferably at 0° C. to +40° C. The reaction can be carried out under atmospheric, elevated or reduced pressure (e.g. from 0.5 to 5 bar). It is generally carried out under atmospheric pressure.

Inert solvents for process step (IX)→(I-A) are for example ethers such as diethyl ether, tert-butyl methyl ether, dioxane, tetrahydrofuran, glycol dimethyl ether or diethylene glycol dimethyl ether, hydrocarbons such as benzene, toluene, xylene, hexane, cyclohexane or petroleum fractions, halohydrocarbons such as dichloromethane or chlorobenzene, or other solvents such as, for example, ethyl acetate or dimethylformamide. It is likewise possible to employ mixtures of the solvents mentioned. Tetrahydrofuran is preferred.

Suitable as base are the usual inorganic or organic bases. These include in particular alkali metal carbonates such as sodium, potassium or cesium carbonate, or else phosphazene bases such as, for example, 1-tert-butyl-2,2,4,4,4-pentakis(dimethylamino)-2⁵,4⁵-catenadiphosphazene (phosphazene base P₂-tert-Bu). Cesium carbonate or the phosphazene base P₂-tert-Bu are preferably used.

The reaction generally takes place in a temperature range from −40° C. to +100° C., preferably at 0° C. to +40° C. The reaction can be carried out under atmospheric, elevated or reduced pressure (e.g. from 0.5 to 5 bar). It is generally carried out under atmospheric pressure.

Inert solvents for process step (I-A)→(I-B) are for example ethers such as diethyl ether, dioxane, tetrahydrofuran, glycol dimethyl ether or diethylene glycol dimethyl ether, alcohols such as methanol, ethanol, n-propanol, iso-propanol or n-butanol, or dipolar aprotic solvents such as acetone, dimethylformamide, dimethyl sulfoxide or acetonitrile, or else water. It is likewise possible to employ mixtures of the solvents mentioned. Dioxane/water mixtures are preferably used.

Suitable acids are aqueous solutions of the usual inorganic acids such as, for example, hydrochloric acid, sulfuric acid, phosphoric acid or hydrobromic acid. Hydrochloric acid is preferred. The reaction generally takes place in a temperature range from +20° C. to +150° C., preferably at +50° C. to +100° C. The reaction can be carried out under atmospheric, elevated or reduced pressure (e.g. from 0.5 to 5 bar). It is generally carried out under atmospheric pressure.

Process step (I-B)→(I-C) [n≠0] is carried out by the abovementioned methods known from the literature for the homologization of carboxylic acids.

Process step (I-B)→(I) or (I-C)→(I) is carried out by methods known from the literature for the esterification or amidation (amide formation) of carboxylic acids.

Inert solvents for an amidation in process step (I-B)+(XI)→(I) or (I-C)+(XI)→(I) are for example ethers such as diethyl ether, tert-butyl methyl ether, dioxane, tetrahydrofuran, glycol dimethyl ether or diethylene glycol dimethyl ether, hydrocarbons such as benzene, toluene, xylene, hexane, cyclohexane or petroleum fractions, halohydrocarbons such as dichloromethane, trichloro-methane, tetrachloromethane, 1,2-dichloroethane, trichloroethylene or chlorobenzene, or other solvents such as acetone, ethyl acetate, pyridine, dimethyl sulfoxide, dimethylformamide, N,N-di-methylpropyleneurea (DMPU), N-methylpyrrolidone (NMP) or acetonitrile. It is likewise possible to use mixtures of the solvents mentioned. Tetrahydrofuran, dimethylformamide or mixtures of these two solvents are preferred.

Condensing agents suitable for an amide formation in process step (I-B)+(XI)→(I) or (I-C)+(XI)→(I) are for example carbodiimides such as N,N′-diethyl-, N,N′-dipropyl-, N,N′-diisopropyl-, N,N′-dicyclohexylcarbodiimide (DCC) or N-(3-dimethylaminoisopropyl)-N′-ethylcarbodiimide hydrochloride (EDC), phosgene derivatives such as N,N′-carbonyldiimidazole (CDI), 1,2-oxazolium compounds such as 2-ethyl-5-phenyl-1,2-oxazolium 3-sulfate or 2-tert-butyl-5-methylisoxazolium perchlorate, acylamino compounds such as 2-ethoxy-1-ethoxycarbonyl-1,2-dihydroquinoline, or isobutyl chloroformate, propanephosphonic anhydride, diethyl cyanophosphonate, bis(2-oxo-3-oxa-zolidinyl)phosphoryl chloride, benzotriazol-1-yloxytris(dimethylamino)phosphonium hexafluorophosphate, benzotriazol-1-yloxytris(pyrrolidino)phosphonium hexafluorophosphate (PyBOP), O-(benzotriazol-1-yl)-N,N,N′,N′-tetramethyluronium tetrafluoroborate (TBTU), O-(benzotriazol-1-yl)-N,N,N′,N′-tetramethyluronium hexafluorophosphate (HBTU), 2-(2-oxo-1-(2H)-pyridyl)-1,1,3,3-tetramethyluronium tetrafluoroborate (TPTU), O-(7-azabenzotriazol-1-yl)-N,N,N′,N′-tetramethyluronium hexafluorophosphate (HATU) or O-(1H-6-chlorobenzotriazol-1-yl)-1,1,3,3-tetramethyluronium tetrafluoroborate (TCTU), where appropriate combined with further auxiliaries such as 1-hydroxybenzotriazole (HOBt) or N-hydroxysuccinimide (HOSu), and suitable bases are alkali metal carbonates, e.g. sodium or potassium carbonate or bicarbonate, or organic bases such as trialkylamines, e.g. triethylamine, N-methylmorpholine, N-methylpiperidine or N,N-diisopropylethylamine. PyBOP combined with N,N-diisopropylethylamine is preferably used.

An amide formation in process step (I-B)+(XI)→(I) or (I-C)+(XI)→(I) is generally carried out in a temperature range from 0° C. to +100° C., preferably at 0° C. to +40° C. The reaction can take place under atmospheric, elevated or reduced pressure (e.g. from 0.5 to 5 bar). It is generally carried out under atmospheric pressure.

The compounds of the formula (V) can be prepared in analogy to processes known from the literature by initially converting a compound of the formula (XII)

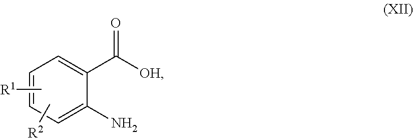

in which R¹ and R² each have the meanings indicated above,
with acetic anhydride into a benzoxazin-4-one derivative of the formula (XIII)

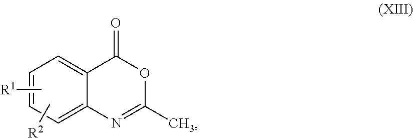

in which R¹ and R² each have the meanings indicated above,
   [cf., for example, Jiang et al., J. Med. Chem. 33 (6), 1721-1728 (1990)], subsequently converting in an inert solvent by reaction with an organometallic compound of the formula (XIV)

A-M tm (XIV),
in which A has the meaning indicated above, and
M is lithium or the Grignard residue —MgCl, —MgBr or —MgI,
and subsequent acidic hydrolysis into a compound of the formula (XV)

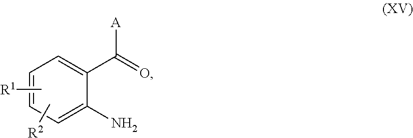

in which A, R¹ and R² each have the meanings indicated above,
   [cf., for example, Miki et al., Bioorg. Med. Chem. 10, 401-414 (2002)] and then converting the latter by diazotization by methods customary in the literature into the compound of the formula (V)

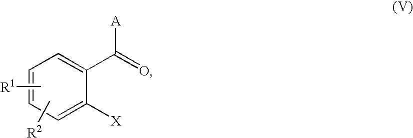

in which A, R¹, R² and X each have the meanings indicated above.

The compounds of the formulae (X), (XI), (XII) and (XIV) are commercially available, known from the literature or can be prepared by methods customary in the literature.

The compounds of the formulae (II) and (III) are commercially available, known from the literature or can be prepared in analogy to processes known from the literature [cf., for example, Brown et al., Tetrahedron Lett. 29, 2631-2634 (1988) and Martin et al., Tetrahedron 53, 8997-9006 (1997)].

The preparation of the compounds of the invention can be illustrated by the following synthesis schemes:

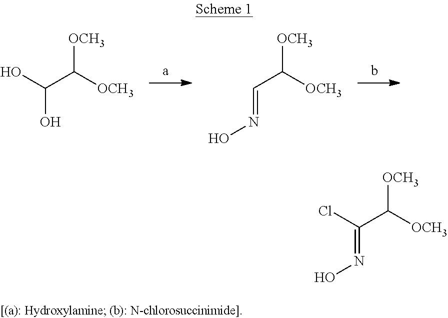

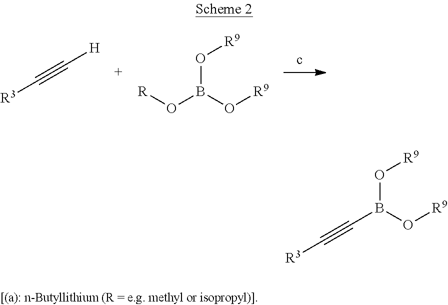

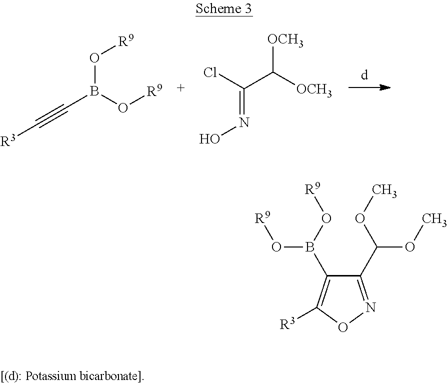

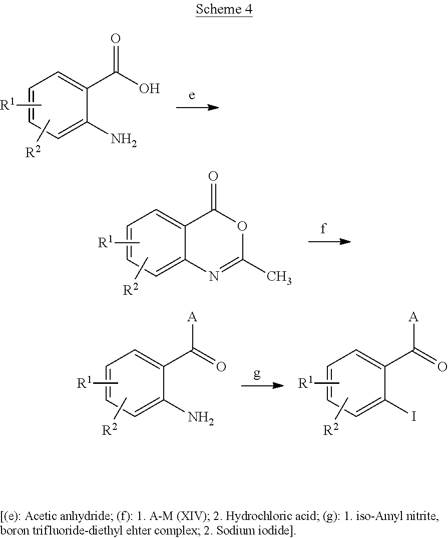

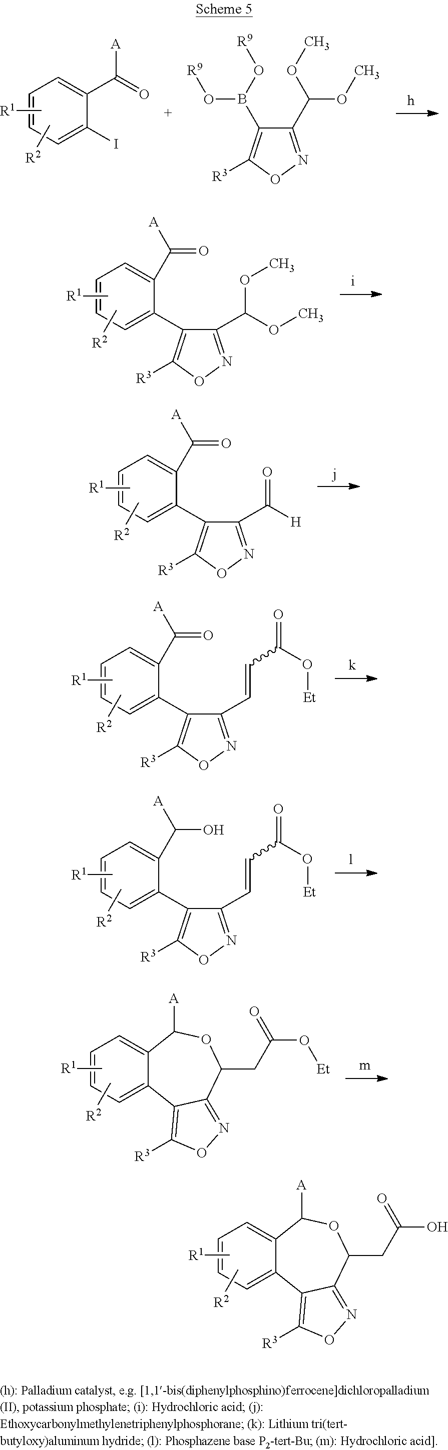

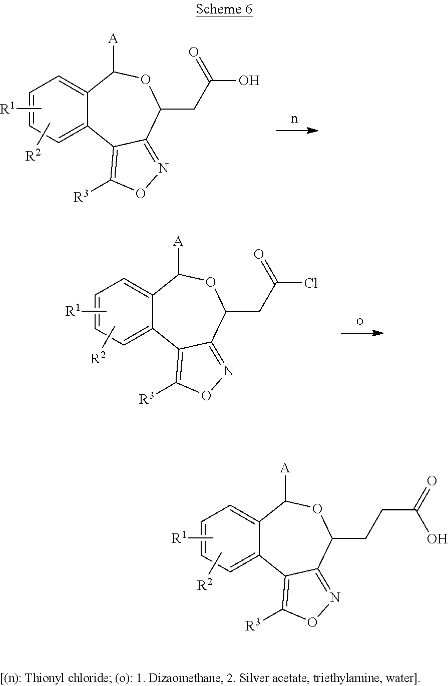

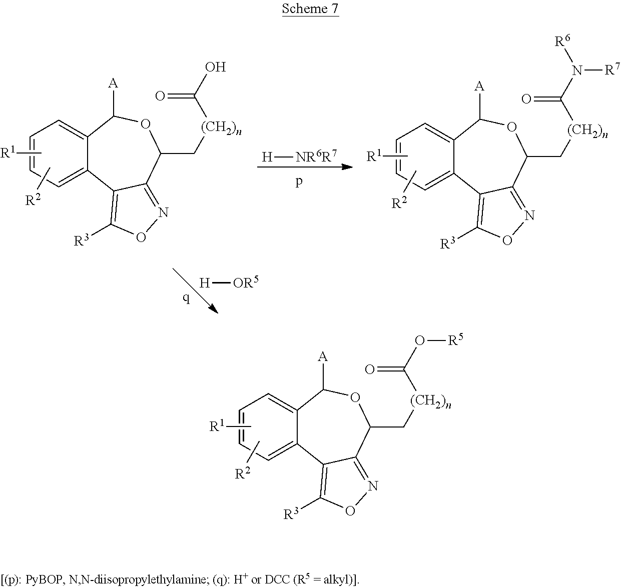

The compounds according to the invention have valuable pharmacological properties and can be used for the prevention and treatment of disorders in humans and animals.

In particular, the compounds according to the invention are highly effective inhibitors of squalene synthase and inhibit cholesterol biosynthesis. The compounds according to the invention bring about a lowering of the cholesterol level and of the triglyceride level in the blood. They can therefore be employed for the treatment and prevention of cardiovascular disorders, in particular of hypolipoproteinemia, dyslipidemias, hyperlipidemias, arteriosclerosis, resterosis and ischemias. The compounds according to the invention may additionally also be used for the treatment and prevention of adiposity and corpulence (obesity). The compounds according to the invention are further suitable for the treatment and prevention of strokes and of Alzheimer's disease.

The present invention further relates to the use of the compounds according to the invention for the treatment and/or prophylaxis of disorders, in particular of the aforementioned disorders.

The present invention further relates to the use of the compounds according to the invention for producing a medicament for the treatment and/or prophylaxis of disorders, especially of the aforementioned disorders.

The present invention further relates to a method for the treatment and/or prophylaxis of disorders, in particular of the aforementioned disorders, using an effective amount of at least one of the compounds according to the invention.

The present invention further relates to medicaments comprising at least one compound according to the invention and at least one or more further active ingredients, in particular for the treatment and/or prophylaxis of the aforementioned disorders. Examples which may be preferably mentioned of active ingredients suitable for combination are: cholesterol-lowering statins, cholesterol absorption inhibitors, HDL-elevating or triglyceride-lowering and/or apolipoprotein B-lowering substances, oxidation inhibitors or compounds having antiinflammatory activity.

Combinations with these active ingredients are preferably suitable for the treatment of dyslipidemias, combined hyperlipidemias, hypercholesterolemias or hypertriglyceridemias. Said combinations can also be employed for the primary or secondary prevention of coronary heart disease (e.g. myocardial infarction) and for peripheral arterial disorders.

Examples of statins in the context of the invention are lovastatin, simvastatin, pravastatin, fluvastatin, atorvastatin, rosuvastatin and pitavastatin. Examples of cholesterol absorption inhibitors are cholestyramines or ezetimibe; examples of HDL-elevating or triglyceride-lowering or apolipoprotein B-lowering substances are fibrates, niacin, PPAR agonists, IBAT inhibitors, MTP inhibitors and CETP inhibitors. Compounds having antiinflammatory activity are, for example, aspirin.

The present invention further relates additionally to the combination of the compounds according to the invention with a glucosidase inhibitor and/or amylase inhibitor for the treatment of familial hyperlipidemia, of adiposity (obesity) and of diabetes mellitus.

Examples of glucosidase inhibitors and/or amylase inhibitors in the context of the invention are acarbose, adiposins, voglibose, miglitol, emiglitates, MDL-25637, camiglibose (MDL-73945), tendamistats, AI-3688, trestatin, pradimicin Q and salbostatin. Combination of acarbose, miglitol, emiglitates or voglibose with one of the compounds according to the invention is preferred.

The compounds of the invention can act systemically and/or locally. For this purpose, they can be administered in a suitable way such as, for example, by the oral, parenteral, pulmonal, nasal, sublingual, lingual, buccal, rectal, dermal, transdermal, conjunctival or otic route or as implant or stent.

The compounds of the invention can be administered in administration forms suitable for these administration routes.

Suitable for oral administration are administration forms which function according to the prior art and deliver the compounds of the invention rapidly and/or in modified fashion, and which contain the compounds of the invention in crystalline and/or amorphized and/or dissolved form, such as, for example, tablets (uncoated or coated tablets, for example having enteric coatings or coatings which are insoluble or dissolve with a delay and control the release of the compound according to the invention), tablets which disintegrate rapidly in the mouth, or films/wafers, films/lyophilizates, capsules (for example hard or soft gelatin capsules), sugar-coated tablets, granules, pellets, powders, emulsions, suspensions, aerosols or solutions.

Parenteral administration can take place with avoidance of an absorption step (e.g. intravenous, intraarterial, intracardiac, intraspinal or intralumbar) or with inclusion of an absorption (e.g. intramuscular, subcutaneous, intracutaneous, percutaneous or intraperitoneal). Administration forms suitable for parenteral administration are, inter alia, preparations for injection and infusion in the form of solutions, suspensions, emulsions, lyophilizates or sterile powders.

Suitable for the other administration routes are, for example, pharmaceutical forms for inhalation (inter alia powder inhalers, nebulizers), nasal drops, solutions or sprays; tablets for lingual, sublingual or buccal administration, films/wafers or capsules, suppositories, preparations for the ears or eyes, vaginal capsules, aqueous suspensions (lotions, shaking mixtures), lipophilic suspensions, ointments, creams, transdermal therapeutic systems (e.g. patches), milk, pastes, foams, dusting powders, implants or stents.

Oral or parenteral administration is preferred, especially oral or intravenous administration.

The compounds of the invention can be converted into the stated administration forms. This can take place in a manner known per se by mixing with inert, nontoxic, pharmaceutically suitable excipients. These excipients include, inter alia, carriers (for example microcrystalline cellulose, lactose, mannitol), solvents (e.g. liquid polyethylene glycols), emulsifiers and dispersants or wetting agents (for example sodium dodecyl sulfate, polyoxysorbitan oleate), binders (for example polyvinylpyrrolidone), synthetic and natural polymers (for example albumin), stabilizers (e.g. antioxidants such as, for example, ascorbic acid), colors (e.g. inorganic pigments such as, for example, iron oxides) and masking flavors and/or odors.

The present invention further relates to medicaments which comprise at least one compound according to the invention, normally together with one or more inert, nontoxic, pharmaceutically suitable excipients, and to the use thereof for the aforementioned purposes.

It has generally proved advantageous to administer on parenteral administration amounts of about 0.001 to 1 mg/kg, preferably about 0.01 to 0.5 mg/kg, of body weight to achieve effective results, and on oral administration the dosage is about 0.01 to 100 mg/kg, preferably about 0.01 to 20 mg/kg, and very particularly preferably 0.1 to 10 mg/kg, of body weight.

It may nevertheless be necessary where appropriate to deviate from the stated amounts, in particular as a function of the body weight, route of administration, individual response to the active ingredient, nature of the preparation and time or interval over which administration takes place. Thus, it may be sufficient in some cases to make do with less than the aforementioned minimum amount, whereas in other cases the stated upper limit must be exceeded. It may in the event of administration of larger amounts be advisable to divide these into a plurality of individual doses over the day.

The following exemplary embodiments illustrate the invention. The invention is not restricted to the examples.

The percentage data in the following tests and examples are, unless indicated otherwise, percentages by weight; parts are parts by weight. Solvent ratios, dilution ratios and concentration data for the liquid/liquid solutions are in each case based on volume.

### A. EXAMPLES

Abbreviations and Acronyms:
b.p. boiling point
CI chemical ionization (in MS)
conc. concentrated
DCC N,N′-Dicyclohexylcarbodiimide
DMF N,N-Dimethylformamide
DMSO Dimethyl sulfoxide
ee Enantiomeric excess
EI Electron impact ionization (in MS)
ESI Electrospray ionization (in MS)
Et Ethyl
GC/MS Coupled gas chromatography-mass spectrometry
h hour(s)
HPLC High pressure, high performance liquid chromatography
LC/MS Coupled liquid chromatography-mass spectrometry
min. minute(s)
MS Mass spectrometry
NMR Nuclear magnetic resonance spectrometry
Nonaflate Nonafluorobutanesulfonate
PyBOP Benzotriazol-1-yloxy-tris(pyrrolidino)phosphonium hexafluorophosphate
rac racemic
RT Room temperature
Rₜ Retention time (in HPLC)
TFA Trifluoroacetic acid
THF Tetrahydrofuran
Tresylate 2,2,2-Trifluoroethanesulfonate
Triflate Trifluoromethanesulfonate
v/v Volume to volume ratio (of a solution)

LC/MS, GC/MS and HPLC Methods:

Method 1:

Instrument: Micromass GCT, GC 6890; column: Restek RTX-35MS, 30 m×250 μm×0.25 μm; constant flow with helium: 0.88 mumin.; oven: 60° C.; inlet: 250° C.; gradient: 60° C. (hold for 0.30 min), 50° C./min.→120° C., 16° C./min.→250° C., 30° C./min.→300° C. (hold for 1.7 min).

Method 2:

Instrument: Micromass Quattro LCZ with HPLC Agilent Series 1100; column: Phenomenex Synergi 2μ Hydro-RP Mercury, 20 mm×4 mm; eluent A: 1 l water+0.5 ml 50% formic acid, eluent B: 1 l acetonitrile+0.5 ml 50% formic acid; gradient: 0.0 min. 90% A→2.5 min. 30% A→3.0 min. 5% A→4.5 min. 5% A; flow rate: 0.0 min. 1 ml/min.→2.5 min./3.0 min./4.5 min. 2 ml/min.; oven: 50° C.; UV detection: 208-400 nm.

Method 3:

MS instrument type: Micromass ZQ; HPLC instrument type: Waters Alliance 2795; column: Phenomenex Synergi 2μ Hydro-RP Mercury, 20 mm×4 mm; eluent A: 1 l water+0.5 ml 50% formic acid, eluent B: 1 l acetonitrile+0.5 ml 50% formic acid; gradient: 0.0 min. 90% A→2.5 min. 30% A→3.0 min. 5% A→4.5 min. 5% A; flow rate: 0.0 min. 1 ml/min.→2.5 min./3.0 min./4.5 min. 2 ml/min.; oven: 50° C.; UV detection: 210 nm.

Method 4:

MS instrument type: Micromass ZQ; HPLC instrument type: HP 1100 Series; UV DAD; column: Phenomenex Synergi 2μ Hydro-RP Mercury, 20 mm×4 mm; eluent A: 1 l water+0.5 ml 50% formic acid, eluent B: 1 l acetonitrile+0.5 ml 50% formic acid; gradient: 0.0 min. 90% A→2.5 min. 30% A→3.0 min. 5% A→4.5 min. 5% A; flow rate: 0.0 min. 1 ml/min.→2.5 min./3.0 min./4.5 min. 2 ml/min.; oven: 50° C.; UV detection: 210 nm.

Starting Compounds and Intermediates:

### Example 1A

### N-Hydroxy-2,2-dimethoxyethanimidoyl chloride

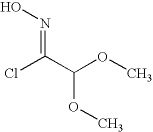

6.608 g of hydroxylamine hydrochloride (95.10 mmol) dissolved in 110 ml of methanol are added dropwise to 22.7 ml of a 25% strength methanolic sodium methoxide solution (95.10 mmol) at 10° C. The reaction mixture is stirred at 10° C. for 1 h, and the resulting precipitate is filtered off and washed with a little methanol. The combined filtrates are mixed with 20 g (86.5 mmol) of a 45% strength solution of glyoxal 1,1-dimethyl acetal in tert-butyl methyl ether and stirred at room temperature for 16 h. For working up, 50 ml of water are added, the methanol is removed in a rotary evaporator, and the residue is extracted four times with dichloromethane. The combined organic phases are dried over sodium sulfate and concentrated in a rotary evaporator. Drying under a high vacuum results in 6.19 g of an oily residue which is employed without further purification for the subsequent reaction.

The resulting residue is dissolved in 50 ml of DMF and, at room temperature, 7.772 g of N-chloro-succinimide (58.20 mmol) are added in portions. After the reaction starts, an ice/acetone cooling mixture is used for cooling in such a way that the temperature of the reaction mixture does not exceed +40° C. After the temperature of the mixture has returned to room temperature, the cooling bath is removed and stirring is continued for 2 h. For working up, 300 ml of cold water (approx. 5° C.) are added, and the mixture is extracted three times with tert-butyl methyl ether. The combined organic phases are washed twice with water, dried over sodium sulfate and concentrated in a rotary evaporator. The residue after drying is dissolved in 10 ml of ethyl acetate, and cyclohexane is slowly added (about 40 ml) until crystallization of the product starts. To complete the crystallization, the mixture is stored at 5° C. for 16 h. The resulting precipitate is filtered off and dried under high vacuum. 3.26 g (41% of theory) of the title compound are obtained.

¹H-NMR (400 MHz, CDCl₃): δ=3.44 (s, 6H), 4.89 (s, 1H), 7.86 (s, 1H).

MS (EI): m/z (rel. Int. %)=75 (100) [M−78]⁺, 122 (48) [M−OCH₃]⁺.

### Example 2A

### 4,4,5,5-Tetramethyl-2-(3-methylbut-1-yn-1-yl)-1,3,2-dioxaborolane

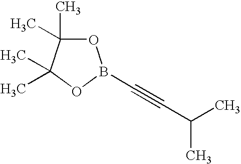

Under an argon atmosphere, 5.00 g of 3-methyl-1-butyne (70.47 mmol) are dissolved in 60 ml of THF and, at −78° C., 44 ml of a solution of n-butyllithium in hexane (1.6 M, 70.47 mmol) are added dropwise. Subsequently 13.11 g of 2-isopropoxy-4,4,5,5-tetramethyl-1,3,2-dioxaborolane (70.47 mmol), dissolved in 60 ml of THF, are added dropwise at −78° C. The reaction mixture is stirred at −78° C. for 2 h and then, for working up, 70 ml of a 1 N solution of hydrogen chloride in diethyl ether are added dropwise. The mixture is warmed to room temperature and concentrated in a rotary evaporator. The residue is stirred with 50 ml of diethyl ether, and the precipitate is filtered off and washed twice with 10 ml of diethyl ether. The combined filtrates are concentrated in a rotary evaporator, and the residue is fractionally distilled under high vacuum. 10.51 g (77% of theory) of the title compound are obtained as a colorless liquid (b.p. 48-50° C./1.4 mbar).

¹H-NMR (400 MHz, CDCl₃): δ=1.19 (d, J=6.8, 6H), 1.27 (s, 12H), 2.61 (sept, J=6.8, 1H).

GC/MS (Method 1): Rₜ=5.17 min.; MS (EI): m/z (rel. int. %)=67 (100), 179 (55) [M−CH₃]⁺.

### Example 3A

### 3-(Dimethoxymethyl)-5-isopropyl-4-(4,4,5,5-tetramethyl-1,3,2-dioxaborolan-2-yl)isoxazole

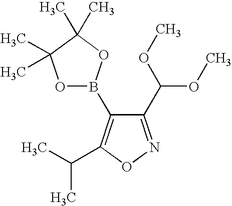

Under an argon atmosphere, 3.703 g of the compound from Example 2A (19.08 mmol) are dissolved in 10 ml of dry 1,2-dimethoxyethane, and 3.820 g of potassium bicarbonate (19.08 mmol) which has previously been dried under high vacuum for 2 h, are added. At 65° C., 2.930 g of the compound from Example 1A, dissolved in 20 ml of 1,2-dimethoxyethane, are very slowly added dropwise by means of a syringe pump over the course of 56 h. The reaction mixture is then stirred at 65° C. for a further 8 h. After cooling, the reaction mixture is filtered and the filtrate is concentrated in a rotary evaporator. The residue is dried under high vacuum and fractionated by preparative HPLC (eluent: acetonitrile/water, gradient 20:80→95:5). The product-containing fractions are combined and lyophilized. 1.00 g (17% of theory) of the title compound is obtained.

¹H-NMR (500 MHz, CDCl₃): δ=1.31 (d, J=6.8, 6H), 1.31 (s, 12H), 3.45 (s, 6H), 3.47 (sept, J=6.8, 1H), 5.72 (s, 1H).

¹³C-NMR (125 MHz, CDCl₃): δ=21.16, 24.91, 27.82, 53.74, 83.74, 98.21, 163.94, 185.77.

LC/MS (Method 4): Rₜ=2.73 min.; MS (ESIpos): m/z=312 [M+H]⁺.

GC/MS (Method 1): Rₜ=9.39 min.; MS (EI): m/z (rel. Int. %)=75 (100), 280 (10) [M−31]⁺.

### Example 4A

### 6-Chloro-2-methyl-4H-3,1-benzoxazin-4-one

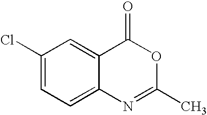

A mixture of 9.42 g of 2-amino-5-chlorobenzoic acid (54.9 mmol) and 31.1 ml of acetic anhydride (33.6 g, 329 mmol) is heated under reflux for 2 h. After cooling, the resulting precipitate is filtered off with suction and washed twice with 50 ml of diethyl ether. 9.01 g (83% of theory) of the product are obtained.

¹H-NMR (300 MHz, CDCl₃): δ=2.47 (s, 3H), 7.50 (d, J=8.7, 1H), 7.74 (dd, J=8.7, 2.3, 1H), 8.15 (d, J=2.3, 1H).

GC/MS (Method 1): Rₜ=8.13 min.; MS (EI): m/z (rel. Int. %)=180 (75), 195 (100) [M]⁺.

### Example 5A

### (2-Amino-5-chlorophenyl)(2,3-dimethoxyphenyl)methanone

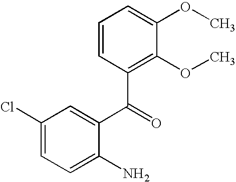

Under argon, 9.07 ml of veratrole (9.28 g, 47.4 mmol) are dissolved in 40 ml of THF. At 0° C., 22.0 ml of n-butyllithium (3.53 g, 55.0 mmol; 1.6 M solution in hexane) are slowly added. After 30 min, this suspension is added at 0° C. to 9.28 g of the compound from Example 4A in 40 ml of THF. After 30 min, the solvent is removed under reduced pressure. The residue is taken up in 48 ml of ethanol and 20 ml of water, 32 ml of concentrated hydrochloric acid are added, and the mixture is heated under reflux for 3 h. 100 ml of water are added, and the mixture is extracted three times with 75 ml of diethyl ether each time. The combined organic phases are washed with 1 N sodium hydroxide solution and with saturated sodium chloride solution (100 ml each), dried over magnesium sulfate and freed of solvent under reduced pressure. The residue is purified by chromatography on a silica gel column (mobile phase: cyclohexane/ethyl acetate 4:1). 6.53 g (47% of theory) of the product are obtained.

¹H-NMR (400 MHz, CDCl₃): δ=3.78 (s, 3H), 3.92 (s, 3H), 6.38 (br. s, 2H), 6.65 (d, J=8.8, 1H), 6.82 (dd, J=7.6, 1.5, 1H), 7.03 (dd, J=8.3, 1.2, 1H), 7.10-7.23 (m, 3H).

LC/MS (Method 4): Rₜ=2.53 min.; MS (ESIpos): m/z=292 [M+H]⁺.

### Example 6A

### (5-Chloro-2-iodophenyl)(2,3-dimethoxyphenyl)methanone

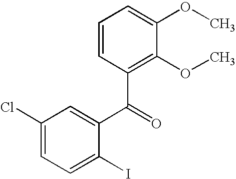

A solution of 10.00 g of (2-amino-5-chlorophenyl)(2,3-dimethoxyphenyl)methanone from example 5A (34.28 mmol in 170 ml of THF is added dropwise to 9.73 g of boron trifluoride-diethyl ether complex (68.56 mmol) at 0° C. At −10° C., 5.22 g of isoamyl nitrite (44.56 mmol), dissolved in 10 ml of THF, are added dropwise to the solution, and the mixture is stirred at −10° C. for 30 min. The resulting diazonium salt is precipitated by adding 100 ml of cold diethyl ether. After filtration, the diazonium salt is added in portions to a solution of 6.68 g of sodium iodide (44.56 mmol) in 170 ml of acetone (gas evolution). The reaction mixture is stirred at room temperature for 4 h, then added to 300 ml of ice-water and extracted three times with dichloromethane. The combined organic phases are dried over sodium sulfate and concentrated in a rotary evaporator. The residue is purified by chromatography on silica gel (mobile phase: cyclohexane/ethyl acetate 20:1). 5.72 g (41% of theory) of the title compound are obtained.

¹H-NMR (400 MHz, CDCl₃): δ=3.55 (s, 3H), 3.89 (s, 3H), 7.09-7.17 (m, 3H), 7.22-7.28 (m, 2H), 7.83 (d, J=8.3, 1H).

LC/MS (Method 2): Rₜ=2.87 min.; MS (ESIpos): m/z=403 [M+H]⁺.

### Example 7A

### {5-Chloro-2-[3-(dimethoxymethyl)-5-isopropylisoxazol-4-yl]phenyl}(2,3-dimethoxyphenyl)-methanone

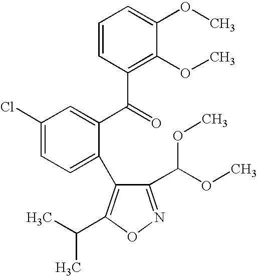

Under an argon atmosphere, 398 mg of the compound from Example 6A (0.988 mmol) are dissolved in 20 ml of dioxane, and 615 mg of the compound from Example 3A (1.976 mmol) are added. Subsequently, 382 mg of potassium phosphate (1.798 mmol) and 161 mg of [1,1′-bis(diphenyl-phosphino)ferrocene]dichloropalladium(II) (1:1 complex with dichloromethane, 0.198 mmol) are added, and the reaction mixture is then stirred at 85° C. for 72 h. After cooling, 15 ml of water are added, and the mixture is extracted three times with dichloromethane. The combined organic phases are washed with saturated sodium chloride solution, dried over sodium sulfate and concentrated in a rotary evaporator. The residue is purified by chromatography on silica gel (mobile phase: cyclohexane/diethyl ether 2:1). 460 mg (42% of theory) of the title compound are obtained.

¹H-NMR (400 MHz, CDCl₃): δ=1.15 (d, J=7.3, 3H), 1.19 (d, J=6.9, 3H), 2.90 (sept, J=7.3, 1H), 3.19 (s, 3H), 3.34 (s, 3H), 3.61 (s, 3H), 3.86 (s, 3H), 5.30 (s, 1H), 6.90-6.92 (m, 1H), 7.00-7.06 (m, 2H), 7.18-7.20 (m, 1H), 7.46-7.52 (m, 2H).

LC/MS (Method 2): Rₜ=2.97 min.; MS (ESIpos): m/z=428 [M−OCH₃]⁺.

MS (CI): m/z=477 [M+NH₄]⁺.

### Example 8A

### Ethyl(2E)-3-{4-[4-chloro-2-(2,3-dimethoxybenzoyl)phenyl]-5-isopropylisoxazol-3-yl}acrylate

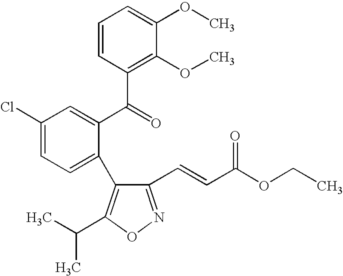

277 mg of the compound from Example 7A (0.602 mmol) are dissolved in 3 ml of THF, and 1.8 ml of 10% strength hydrochloric acid are added. The reaction mixture is heated under reflux for 42 h and, after cooling, water is added, and the mixture is extracted three times with tert-butyl methyl ether. The combined organic phases are washed twice with saturated sodium bicarbonate solution and once with saturated sodium chloride solution, dried over sodium sulfate and concentrated in a rotary evaporator. The oily residue is dissolved in 15 ml of dichloromethane, 214 mg of ethoxy-carbonylmethyltriphenylphosphorane (0.614 mmol) are added, and the reaction mixture is stirred at room temperature for 16 h. The solvent is then removed in a rotary evaporator, and the residue is purified by preparative HPLC (eluent: acetonitrile/water, gradient 20:80→95:5). 213 mg (72% of theory) of the title compound are obtained.

¹H-NMR (300 MHz, CDCl₃): δ=1.15 (d, J=6.9, 3H), 1.21 (d, J=6.9, 3H), 1.29 (t, J=6.9, 3H), 2.89 (sept, J=6.9, 1H), 3.57 (s, 3H), 3.81 (s, 3H), 4.15-4.26 (m, 2H), 6.23 (d, J=16.4, 1H), 6.83-6.86 (m, 1H), 6.97-7.03 (m, 2H), 7.17 (d, J=8.12, 1H), 7.25 (d, J=16.4, 1H), 7.51-7.58 (m, 2H).

LC/MS (Method 4): Rₜ=3.18 min.; MS (ESIpos): m/z=484 [M+H]⁺.

### Exemplary Embodiments

### Example 1

### Ethyl[8-chloro-6-(2,3-dimethoxyphenyl)-1-isopropyl-4H,6H-[2]benzoxepino[4,5-c]isoxazol-4-yl]acetate (Racemic Pair of Diastereomers)

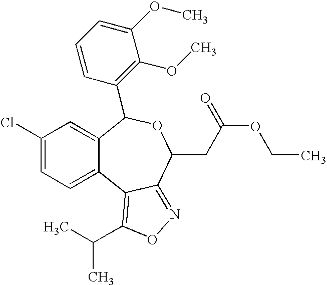

320 mg of the compound from Example 8A (0.661 mmol) are dissolved in 5 ml of dry THF and, at 0° C., 1.47 ml of a 1 M solution of lithium tri(tert-butyloxy)aluminum hydride (1.472 mmol) in THF are added dropwise. The reaction solution is warmed to room temperature while stirring over the course of 2 h. 2 ml of 1 N hydrochloric acid and water are then added, and the mixture is extracted three times with ethyl acetate. The combined organic phases are dried over sodium sulfate and concentrated in a rotary evaporator. The residue is taken up in 10 ml of dry THF and, at 0° C., 0.66 ml of a 2 M solution of 1-tert-butyl-2,2,4,4,4-pentakis(dimethylamino)-2⁵,4⁵-catenadi(phosphazene) (phosphazene base P₂-tert-Bu, 1.320 mmol) in THF is added, and the mixture is stirred at 0° C. for 1 h. 2 ml of 1 N hydrochloric acid and water are added, and the mixture is extracted three times with dichloromethane. The combined organic phases are dried over sodium sulfate and concentrated in a rotary evaporator. The resulting residue is purified by preparative HPLC (eluent: acetonitrile/water, gradient 20:80→95:5). 115 mg (36% of theory) of the title compound are obtained as a mixture of two diastereomers (diastereomer 1-1/diastereomer 1-2 ratio=58:42). For analytical purposes, a small amount is fractionated into the individual diastereomers by repeated preparative HPLC (eluent: acetonitrile/water, gradient 20:80→95:5).

Diastereomer 1-1 (Racemic):

¹H-NMR (400 MHz, CDCl₃): δ=1.18 (t, J=7.1, 3H), 1.39 (d, J=6.9, 3H), 1.52 (d, J=7.1, 3H), 2.84 (dd, J=15.4, 8.8, 1H), 2.95 (dd, J=15.4, 4.2, 1H), 3.47 (sept, J=6.9, 1H), 3.65 (s, 3H), 3.89 (s, 3H), 4.12 (q, J=7.1, 2H), 5.65 (dd, J=8.9, 4.3, 1H), 5.96 (s, 1H), 6.63 (d, J=2.2, 1H), 6.95 (dd, J=8.9, 1.3, 1H), 7.06-7.09 (m, 1H), 7.14 (t, J=7.8, 1H), 7.27-7.29 (m, 1H), 7.37 (d, J=8.3, 1H).

LC/MS (Method 2): Rₜ=3.18 min.; MS (ESIpos): m/z=486 [M+H]⁺.

Diastereomer 1-2 (Racemic):

¹H-NMR (400 MHz, CDCl₃): δ=1.17 (t, J=7.1, 3H), 1.37 (d, J=7.1, 3H), 1.52 (d, J=7.1, 3H), 3.01 (dd, J=15.9, 8.8, 1H), 3.21 (dd, J=15.9, 4.9, 1H), 3.46 (sept, J=7.1, 1H), 3.52 (s, 3H), 3.87 (s, 3H), 4.05-4.14 (m, 2H), 5.37 (dd, J=8.6, 4.9, 1H), 5.90 (s, 1H), 6.89 (d, J=1.7, 1H), 6.94-6.96 (m, 1H), 7.15 (t, J=7.8, 1H), 7.18-7.20 (m, 1H), 7.32 (dd, J=8.1, 2.0, 1H), 7.37 (d, J=8.1, 1H).

LC/MS (Method 4): Rₜ=3.22 min.; MS (ESIpos): m/z=486 [M+H]⁺.

### Example 2

### [8-Chloro-6-(2,3-dimethoxyphenyl)-1-isopropyl-4H,6H-[2]benzoxepino[4,5-c]isoxazol-4-yl]acetic acid (Racemic Pair of Diastereomers)

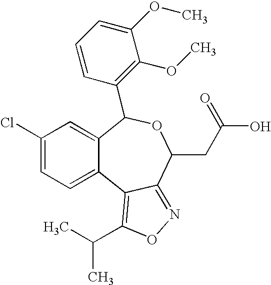

252 mg of the diastereomer mixture from Example 1 (0.519 mmol) are dissolved in 20 ml of dioxane, 3.5 ml of water and 3.5 ml of conc. hydrochloric acid are added, and the mixture is stirred at 80° C. for 19 h. After cooling, the reaction mixture is diluted with 10 ml of water and extracted three times with dichloromethane. The combined organic phases are dried over sodium sulfate and concentrated in a rotary evaporator. The resulting residue is purified by preparative HPLC (eluent: acetonitrile/water, gradient 20:80→95:5). The separated diastereomers of the title compound are obtained.

Diastereomer 2-1 (Racemic):

Yield: 122 mg (51% of theory)

¹H-NMR (300 MHz, CDCl₃): δ=1.40 (d, J=6.8, 3H), 1.53 (d, J=7.0, 3H), 2.93 (dd, J=15.9, 8.7, 1H), 3.04 (dd, J=15.9, 4.2, 1H), 3.47 (sept, J=7.0, 1H), 3.66 (s, 3H), 3.90 (s, 3H), 5.66 (dd, J=8.7, 4.2, 1H), 6.00 (s, 1H), 6.65 (d, J=2.3, 1H), 6.96 (dd, J=8.1, 1.7, 1H), 7.06-7.09 (m, 1H), 7.14-7.18 (m, 1H), 7.30 (dd, J=8.4, 2.4, 1H), 7.38 (d, J=8.1, 1H).

LC/MS (Method 3): Rₜ=2.53 min.; MS (ESIpos): m/z=458 [M+H]⁺.

Diastereomer 2-2 (Racemic):

Yield: 75 mg (31% of theory)

¹H-NMR (400 MHz, CDCl₃): δ=1.38 (d, J=7.0, 3H), 1.53 (d, J=7.0, 3H), 3.08 (dd, J=16.2, 8.1, 1H), 3.30 (dd, J=16.2, 4.9, 1H), 3.47 (sept, J=7.0, 1H), 3.51 (s, 3H), 3.87 (s, 3H), 5.28 (dd, J=8.1, 4.9, 1H), 5.92 (s, 1H), 6.73-6.74 (m, 1H), 6.96 (dd, J=7.8, 1.9, 1H), 7.14-7.36 (m, 4H).

LC/MS (Method 3): Rₜ=2.57 min.; MS (ESIpos): m/z=458 [M+H]⁺.

### Example 3

### [8-Chloro-6-(2,3-dimethoxyphenyl)-1-isopropyl-4H,6H-[2]benzoxepino[4,5-c]isoxazol-4-yl]acetic acid (Separated Stereoisomers)

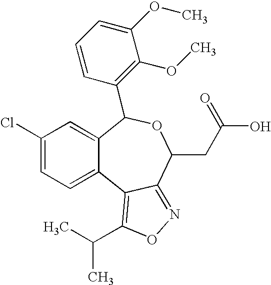

490 mg of a mixture of all the stereoisomers from Example 2 are separated into the four stereoisomers (enantiopure diastereomers) by preparative HPLC on a chiral phase [Agilent 1100 with DAD detection; column: Daicel Chiralpak AD-H, 5 μm, 250 mm×20 mm; eluent A: isohexane, eluent B: isopropanol+0.2% glacial acetic acid+1.0% water; eluent A/B=4:1; flow rate: 15 ml/min.; oven: 25° C.; UV detection: 220 nm]:

Stereoisomer 3-1:

HPLC: Rₜ=4.160 min., proportion of mixture 19.5% [column: Daicel Chiralpak AD-H, 5 μm, 250 mm×4.6 mm; eluent A: isohexane, eluent B: ethanol+0.2% TFA+1.0% water; eluent A/B=4:1; flow rate: 1 mumin.; oven: 25° C.; UV detection: 215 nm]

Yield: 74 mg; content: >96% (215 nm), ee>99.5%

LC/MS (Method 4): Rₜ=2.82 min.; MS (ESIpos): m/z=457 [M+H]⁺.

Stereoisomer 3-2:

HPLC: Rₜ=4.439 min., proportion of mixture 28.5%

Yield: 118 mg; content: >97% (215 nm), ee>99.0%

LC/MS (Method 4): Rₜ=2.78 min.; MS (ESIpos): m/z=457 [M+H]⁺.

Stereoisomer 3-3:

HPLC: Rₜ=6.018 min., proportion of mixture 27.9%

Yield: 101 mg; content: >99% (215 nm), ee>99.0%

LC/MS (Method 4): Rₜ=2.78 min.; MS (ESIpos): m/z=457 [M+H]⁺.

Stereoisomer 3-4:

HPLC: Rₜ=6.610 min., proportion of mixture 19.8%

Yield: 67 mg; content: >98% (215 nm), ee>99.3%

LC/MS (Method 4): Rₜ=2.82 min.; MS (ESIpos): m/z=457 [M+H]⁺.

### Example 4

### Ethyl(1-{[8-chloro-6-(2,3-dimethoxyphenyl)-1-isopropyl-4H,6H-[2]benzoxepino[4,5-c]isoxazol-4-yl]acetyl}piperidin-4-yl)acetate (Stereoisomer 4)

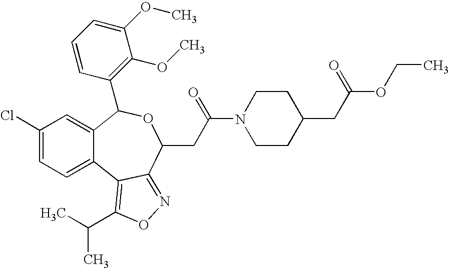

22 mg of stereoisomer 3-4 from Example 3 (0.048 mmol) are dissolved in 1.5 ml of THF, 33 mg of (benzotriazol-1-yloxy)tris(pyrrolidino)phosphonium hexafluorophosphate (PyBOP, 0.062 mmol) and 16 mg of N,N-diisopropylethylamine (0.120 mmol) are added, and the mixture is stirred at room temperature for 30 min. 13 mg of ethyl 4-piperidineacetate hydrochloride (0.062 mmol) are added, and the reaction solution is stirred at room temperature for 16 h. The resulting crude product is purified without further working up directly by preparative HPLC (eluent: acetonitrile/water, gradient 20:80→95:5). 11 mg (37% of theory) of the target compound are obtained.

¹H-NMR (300 MHz, CDCl₃): δ=1.00-2.27 (m, 7H), 1.26 (t, J=7.0, 3H), 1.36 (d, J=7.0, 3H), 1.52 (d, J=7.0, 3H), 2.50-3.22 (m, 4H), 3.41-3.50 (m, 4H), 3.84-3.93 (m, 4H), 4.13 (q, J=7.0, 2H), 4.56-4.65 (m, 1H), 5.27-5.43 (m, 1H), 5.86-5.88 (m, 1H), 6.69-6.72 (m, 1H), 6.93-6.97 (m, 1H), 7.14-7.36 (m, 4H).

LC/MS (Method 3): Rₜ=2.93 min.; MS (ESIpos): m/z=611 [M+H]⁺.

### Example 5

### Ethyl(1-{[8-chloro-6-(2,3-dimethoxyphenyl)-1-isopropyl-4H,6H-[2]benzoxepino[4,5-c]isoxazol-4-yl]acetyl}piperidin-4-yl)acetate (Stereoisomer 3)

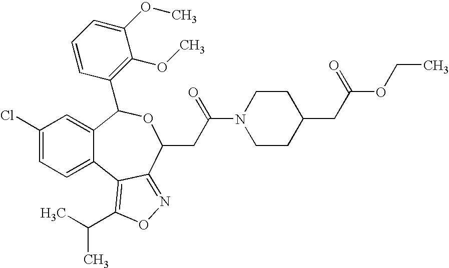

25 mg of stereoisomer 3-3 from Example 3 (0.055 mmol) are dissolved in 2 ml of THF, 37 mg of PyBOP (0.071 mmol) and 18 mg of N,N-diisopropylethylamine (0.136 mmol) are added, and the mixture is stirred at room temperature for 30 min. 15 mg of ethyl 4-piperidineacetate hydrochloride (0.071 mmol) are added, and the reaction solution is stirred at room temperature for 16 h. The resulting crude product is purified without further working up directly by preparative HPLC (eluent: acetonitrile/water, gradient 20:80→95:5). 16 mg (46% of theory) of the target compound are obtained.

¹H-NMR (400 MHz, CDCl₃): δ=1.02-1.75 (m, 4H), 1.25 (t, J=7.2, 3H), 1.40 (d, J=6.8, 3H), 1.47 (d, J=6.8, 3H), 1.95-2.03 (m, 1H), 2.16-2.22 (m, 2H), 2.51-2.59 (m, 1H), 2.89-3.03 (m, 3H), 3.46 (sept, J=6.9, 1H), 3.64 (s, 3H), 3.79-3.89 (m, 1H), 3.87 (s, 3H), 4.13 (q, J=7.2, 2H), 4.58-4.63 (m, 1H), 5.65-5.68 (m, 1H), 6.03-6.07 (m, 1H), 6.71-6.76 (m, 1H), 6.90-6.93 (m, 1H), 6.95-7.01 (m, 1H), 7.07-7.11 (m, 1H), 7.27-7.28 (m, 1H), 7.35 (d, J=8.2, 1H).

LC/MS (Method 2): Rₜ=3.07 min.; MS (ESIpos): m/z=611 [M+H]⁺.

### Example 6

### Ethyl(1-{[8-chloro-6-(2,3-dimethoxyphenyl)-1-isopropyl-4H,6H-[2]benzoxepino[4,5-c]isoxazol-4-yl]acetyl}piperidin-4-yl)acetate (Stereoisomer 2)

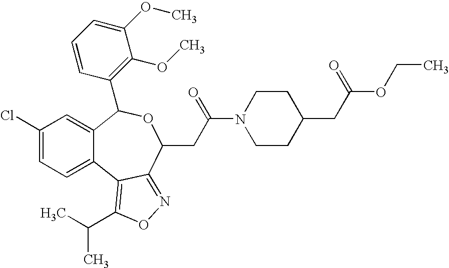

30 mg of stereoisomer 3-2 from Example 3 (0.066 mmol) are dissolved in 4 ml of THF, 44 mg of PyBOP (0.085 mmol) and 11 mg of N,N-diisopropylethylamine (0.085 mmol) are added, and the mixture is stirred at room temperature for 30 min. 15 mg of ethyl 4-piperidineacetate (0.085 mmol) are added, and the reaction solution is stirred at room temperature for 16 h. The resulting crude product is purified without further working up directly by preparative HPLC (eluent: aceto-nitrile/water, gradient 20:80→95:5). 13 mg (32% of theory) of the target compound are obtained.

¹H-NMR (400 MHz, CDCl₃): δ=1.02-1.75 (m, 4H), 1.25 (t, J=7.2, 3H), 1.40 (d, J=6.8, 3H), 1.47 (d, J=6.8, 3H), 1.95-2.03 (m, 1H), 2.16-2.22 (m, 2H), 2.51-2.59 (m, 1H), 2.89-3.03 (m, 3H), 3.46 (sept, J=6.9, 1H), 3.64 (s, 3H), 3.79-3.89 (m, 1H), 3.87 (s, 3H), 4.13 (q, J=7.2, 2H), 4.58-4.63 (m, 1H), 5.65-5.68 (m, 1H), 6.03-6.07 (m, 1H), 6.71-6.76 (m, 1H), 6.90-6.93 (m, 1H), 6.95-7.01 (m, 1H), 7.07-7.11 (m, 1H), 7.27-7.28 (m, 1H), 7.35 (d, J=8.2, 1H).

LC/MS (Method 2): Rₜ=3.06 min.; MS (ESIpos): m/z=611 [M+H]⁺.

### Example 7

### (1-{[8-Chloro-6-(2,3-dimethoxyphenyl)-1-isopropyl-4H,6H-[2]benzoxepino[4,5-c]isoxazol-4-yl]-acetyl}piperidin-4-yl)acetic acid (Stereoisomer 4)

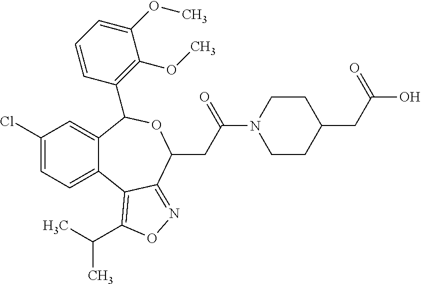

8 mg of the compound from Example 4 (0.012 mmol) are dissolved in 2 ml of dioxane, and 0.2 ml of conc. hydrochloric acid is added. The reaction mixture is stirred at 60° C. for 16 h. For working up, water is added, the mixture is extracted three times with dichloromethane, the combined organic phases are dried over sodium sulfate, and the solvent is removed in a rotary evaporator. The resulting residue is purified by preparative HPLC (eluent: acetonitrile/water with 0.1% formic acid, gradient 20:80→95:5). 5 mg (66% of theory) of the target compound are obtained. LC/MS (Method 2): Rₜ=2.63 min.; MS (ESIpos): m/z=583 [M+H]⁺.

### Example 8

### (1-{[8-Chloro-6-(2,3-dimethoxyphenyl)-1-isopropyl-4H,6H-[2]benzoxepino[4,5-c]isoxazol-4-yl]-acetyl}piperidin-4-yl)acetic acid (Stereoisomer 3)

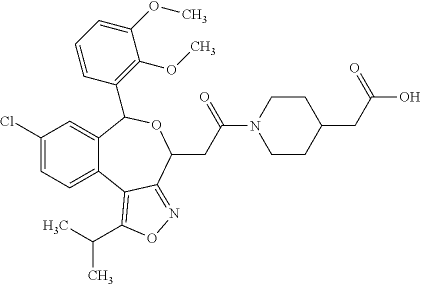

14 mg of the compound from Example 5 (0.023 mmol) are dissolved in 2 ml of dioxane, and 0.2 ml of conc. hydrochloric acid is added. The reaction mixture is stirred at 60° C. for 16 h. For working up, water is added, the mixture is extracted three times with dichloromethane, the combined organic phases are dried over sodium sulfate, and the solvent is removed in a rotary evaporator. The resulting residue is purified by preparative HPLC (eluent: acetonitrile/water with 0.1% formic acid, gradient 20:80→95:5). 5 mg (39% of theory) of the target compound are obtained.

LC/MS (Method 2): Rₜ=2.60 min.; MS (ESIpos): m/z=583 [M+H]⁺.

### Example 9

### (1-{[8-Chloro-6-(2,3-dimethoxyphenyl)-1-isopropyl-4H,6H-[2]benzoxepino[4,5-c]isoxazol-4-yl]-acetyl}piperidin-4-yl)acetic acid (Stereoisomer 2)

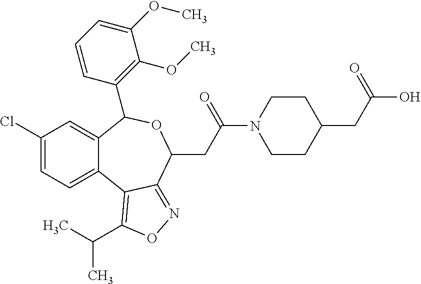

10 mg of the compound from example 6 (0.016 mmol) are dissolved in 1 ml of dioxane, and 0.1 ml of conc. hydrochloric acid is added. The reaction mixture is stirred at 60° C. for 16 h. For working up, water is added, the mixture is extracted three times with dichloromethane, the combined organic phases are dried over sodium sulfate, and the solvent is removed in a rotary evaporator. The resulting residue is purified by preparative HPLC (eluent: acetonitrile/water with 0.1% formic acid, gradient 20:80→95:5). 3 mg (30% of theory) of the target compound are obtained.

LC/MS (Method 3): Rₜ=2.45 min.; MS (ESIpos): m/z=583 [M+H]⁺.

### Example 10

### Ethyl 1-{[8-chloro-6-(2,3-dimethoxyphenyl)-1-isopropyl-4H,6H-[2]benzoxepino[4,5-c]isoxazol-4-yl]acetyl}piperidine-4-carboxylate (Stereoisomer 4)

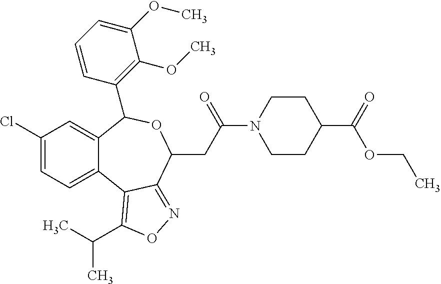

22 mg of stereoisomer 3-4 from Example 3 (0.048 mmol) are dissolved in 1.5 ml of THF, 33 mg of PyBOP (0.062 mmol) and 8 mg of N,N-diisopropylethylamine (0.062 mmol) are added, and the mixture is stirred at room temperature for 30 min. 13 mg of ethyl piperidine-4-carboxylate (0.062 mmol) are added, and the reaction solution is stirred at room temperature for 16 h. The resulting crude product is purified without further working up directly by preparative HPLC (eluent: acetonitrile/water, gradient 20:80→95:5). 20 mg (69% of theory) of the target compound are obtained.

¹H-NMR (300 MHz, CDCl₃): δ=1.26 (t, J=7.2, 3H), 1.36 (d, J=7.0, 3H), 1.42-1.94 (m, 4H), 1.51 (d, J=7.0, 3H), 2.41-2.51 (m, 1H), 2.75-3.23 (m, 4H), 3.44 (s, 3H), 3.46 (sept, J=7.0, 1H), 3.81-3.87 (m, 1H), 3.86 (s, 3H), 4.14 (q, J=7.2, 2H), 4.32-4.46 (m, 1H), 5.32 (dd, J=13.0, 7.0, 1H), 5.86 (s, 1H), 6.71-6.73 (m, 1H), 6.91-6.96 (m, 1H), 7.14-7.19 (m, 1H), 7.24-7.35 (m, 3H).

LC/MS (Method 3): Rₜ=2.90 min.; MS (ESIpos): m/z=597 [M+H]⁺.

### Example 11

### Ethyl 1-{[8-chloro-6-(2,3-dimethoxyphenyl)-1-isopropyl-4H,6H-[2]benzoxepino[4,5-c]isoxazol-4-yl]acetyl}piperidine-4-carboxylate (Stereoisomer 3)

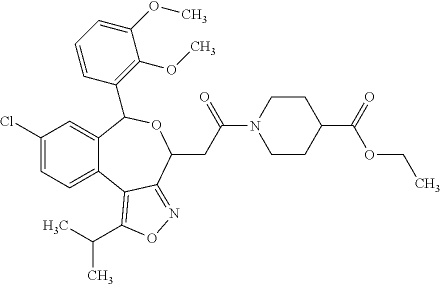

25 mg of stereoisomer 3-3 from Example 3 (0.055 mmol) are dissolved in 1.5 ml of THF, 37 mg of PyBOP (0.071 mmol) and 9 mg of N,N-diisopropylethylamine (0.071 mmol) are added, and the mixture is stirred at room temperature for 30 min. 11 mg of ethyl piperidine-4-carboxylate (0.071 mmol) are added, and the reaction solution is stirred at room temperature for 16 h. The resulting crude product is purified without further working up directly by preparative HPLC (eluent: acetonitrile/water, gradient 20:80→95:5). 21 mg (65% of theory) of the target compound are obtained.

¹H-NMR (300 MHz, CDCl₃): δ=1.25 (t, J=7.2, 3H), 1.40 (d, J=6.8, 3H), 1.48 (d, J=7.0, 3H), 1.52-1.95 (m, 4H), 2.43-2.54 (m, 1H), 2.75-3.13 (m, 4H), 3.46 (sept, J=6.8, 1H), 3.64 (s, 3H), 3.76-3.85 (m, 1H), 3.87 (s, 3H), 4.10-4.18 (m, 2H), 4.36-4.45 (m, 1H), 5.65-5.71 (m, 1H), 6.05 (s, 1H), 6.73-6.74 (m, 1H), 6.91 (dd, J=8.1, 1.3, 1H), 6.95-7.01 (m, 1H), 7.06-7.12 (m, 1H), 7.27-7.36 (m, 2H).

LC/MS (Method 4): Rₜ=3.10 min.; MS (ESIpos): m/z=597 [M+H]⁺.

### Example 12

### Ethyl 1-{[8-chloro-6-(2,3-dimethoxyphenyl)-1-isopropyl-4H,6H-[2]benzoxepino[4,5-c]isoxazol-4-yl]acetyl}piperidine-4-carboxylate (Stereoisomer 2)

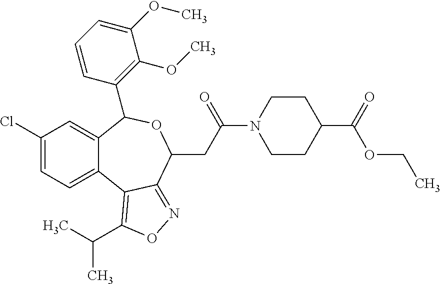

26 mg of stereoisomer 3-2 from Example 3 (0.057 mmol) are dissolved in 1.9 ml of THF, 38 mg of PyBOP (0.074 mmol) and 10 mg of N,N-diisopropylethylamine (0.074 mmol) are added, and the mixture is stirred at room temperature for 30 min. 12 mg of ethyl piperidine-4-carboxylate (0.074 mmol) are added, and the reaction solution is stirred at room temperature for 16 h. The resulting crude product is purified without further working up directly by preparative HPLC (eluent: acetonitrile/water, gradient 20:80→95:5). 21 mg (65% of theory) of the target compound are obtained.

¹H-NMR (300 MHz, CDCl₃): δ=1.25 (t, J=7.2, 3H), 1.40 (d, J=6.8, 3H), 1.48 (d, J=7.0, 3H), 1.52-1.95 (m, 4H), 2.43-2.54 (m, 1H), 2.75-3.13 (m, 4H), 3.46 (sept, J=6.8, 1H), 3.64 (s, 3H), 3.76-3.85 (m, 1H), 3.87 (s, 3H), 4.10-4.18 (m, 2H), 4.36-4.45 (m, 1H), 5.65-5.71 (m, 1H), 6.05 (s, 1H), 6.73-6.74 (m, 1H), 6.91 (dd, J=8.1, 1.3, 1H), 6.95-7.01 (m, 1H), 7.06-7.12 (m, 1H), 7.27-7.36 (m, 2H).

LC/MS (Method 2): Rₜ=3.02 min.; MS (ESIpos): m/z=597 [M+H]⁺.

### Example 13

### 1-{[8-Chloro-6-(2,3-dimethoxyphenyl)-1-isopropyl-4H,6H-[2]benzoxepino[4,5-c]isoxazol-4-yl]-acetyl}piperidine-4-carboxylic acid (Stereoisomer 4)

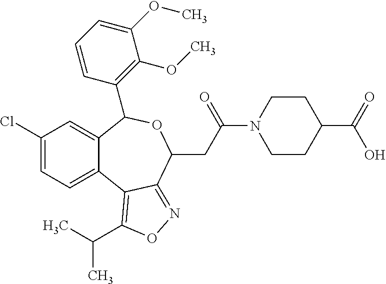

16 mg of the compound from example 10 (0.027 mmol) are dissolved in 2 ml of dioxane, and 0.2 ml of conc. hydrochloric acid is added. The reaction mixture is stirred at 60° C. for 16 h. For working up, water is added, the mixture is extracted three times with dichloromethane, the combined organic phases are dried over sodium sulfate, and the solvent is removed in a rotary evaporator. The resulting residue is purified by preparative HPLC (eluent: acetonitrile/water with 0.1% formic acid, gradient 20:80→95:5). 12 mg (75% of theory) of the target compound are obtained.

¹H-NMR (400 MHz, CDCl₃): δ=1.36 (d, J=6.9, 3H), 1.48-1.98 (m, 4H), 1.51 (d, J=6.9, 3H), 2.49-2.58 (m, 1H), 2.79-3.02 (m, 2H), 3.07-3.20 (m, 2H), 3.44 (s, 3H), 3.46 (sept, J=6.9, 1H), 3.84-3.89 (m, 4H), 4.32-4.46 (m, 1H), 5.23-5.37 (m, 1H), 5.87 (s, 1H), 6.71-6.73 (m, 1H), 6.91-6.95 (m, 1H), 7.14-7.18 (m, 1H), 7.24-7.35 (m, 3H).

LC/MS (Method 2): Rₜ=2.59 min.; MS (ESIpos): m/z=569 [M+H]⁺.

### Example 14

### 1-{[8-Chloro-6-(2,3-dimethoxyphenyl)-1-isopropyl-4H,6H-[2]benzoxepino[4,5-c]isoxazol-4-yl]-acetyl}piperidine-4-carboxylic acid (Stereoisomer 3)

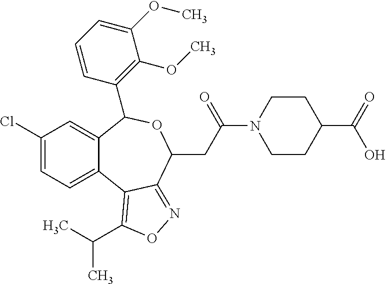

20 mg of the compound from example 11 (0.033 mmol) are dissolved in 2 ml of dioxane, and 0.2 ml of conc. hydrochloric acid is added. The reaction mixture is stirred at 60° C. for 16 h. For working up, water is added, the mixture is extracted three times with dichloromethane, the combined organic phases are dried over sodium sulfate, and the solvent is removed in a rotary evaporator. The resulting residue is purified by preparative HPLC (eluent: acetonitrile/water with 0.1% formic acid, gradient 20:80→95:5). 12 mg (64% of theory) of the target compound are obtained.

¹H-NMR (400 MHz, CDCl₃): δ=1.40 (d, J=6.8, 3H), 1.48 (d, J=6.8, 3H), 1.52-1.99 (m, 4H), 2.50-2.62 (m, 1H), 2.77-3.17 (m, 4H), 3.46 (sept, J=7.0, 1H), 3.64 (s, 3H), 3.70-3.86 (m, 1H), 3.87 (s, 3H), 4.36-4.46 (m, 1H), 5.65-5.71 (m, 1H), 6.05 (s, 1H), 6.73 (s, 1H), 6.90-7.00 (m, 2H), 7.07-7.12 (m, 1H), 7.24-7.35 (m, 2H).

LC/MS (Method 4): Rₜ=2.61 min.; MS (ESIpos): m/z=569 [M+H]⁺.

### Example 15

### 1-{[8-Chloro-6-(2,3-dimethoxyphenyl)-1-isopropyl-4H,6H-[2]benzoxepino[4,5-c]isoxazol-4-yl]-acetyl}piperidin-4-ol (Stereoisomer 3)

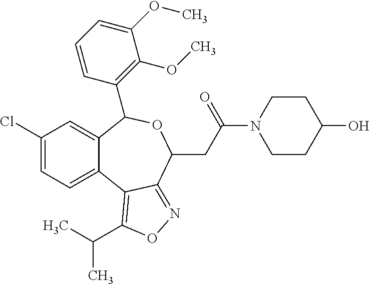

25 mg of stereoisomer 3-3 from Example 3 (0.055 mmol) are dissolved in 1.5 ml of THF, 37 mg of PyBOP (0.071 mmol) and 9 mg of N,N-diisopropylethylamine (0.071 mmol) are added, and the mixture is stirred at room temperature for 30 min. 7 mg of 4-hydroxypiperidine (0.071 mmol) are added, and the reaction solution is stirred at room temperature for 16 h. The resulting crude product is purified without further working up directly by preparative HPLC (eluent: acetonitrile/water, gradient 20:80→95:5). 15 mg (49% of theory) of the target compound are obtained.

¹H-NMR (400 MHz, CDCl₃): δ=1.39-1.49 (m, 9H), 1.77-1.88 (m, 2H), 2.91-3.06 (m, 2H), 3.12-3.23 (m, 2H), 3.43-3.50 (m, 1H), 3.63-3.67 (m, 3H), 3.68-3.77 (m, 1H), 3.86-3.90 (m, 4H), 4.05-4.15 (m, 1H), 5.63-5.69 (m, 1H), 6.05-6.09 (m, 1H), 6.74-6.77 (m, 1H), 6.89-6.92 (m, 1H), 6.95-6.98 (m, 1H), 7.06-7.10 (m, 1H), 7.25-7.36 (m, 2H).

LC/MS (Method 3): Rₜ=2.37 min.; MS (ESIpos): m/z=541 [M+H]⁺.

### Example 16

### 1-{[8-Chloro-6-(2,3-dimethoxyphenyl)-1-isopropyl-4H,6H-[2]benzoxepino[4,5-c]isoxazol-4-yl]-acetyl}piperidin-4-ol (Stereoisomer 2)

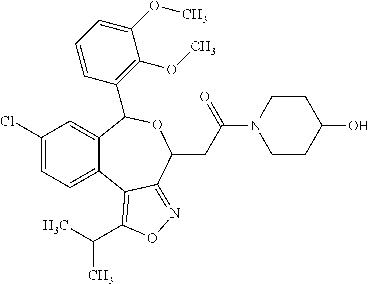

20 mg of stereoisomer 3-2 from Example 3 (0.044 mmol) are dissolved in 3 ml of THF, 30 mg of PyBOP (0.057 mmol) and 7 mg of N,N-diisopropylethylamine (0.057 mmol) are added, and the mixture is stirred at room temperature for 30 min. 6 mg of 4-hydroxypiperidine (0.057 mmol) are added, and the reaction solution is stirred at room temperature for 16 h. The resulting crude product is purified without further working up directly by preparative HPLC (eluent: acetonitrile/water, gradient 20:80→95:5). 14 mg (58% of theory) of the target compound are obtained.

¹H-NMR (400 MHz, CDCl₃): δ=1.34 (d, J=6.8, 3H), 1.40 (d, J=6.8, 3H), 1.44-1.53 (m, 2H), 1.69-1.81 (m, 2H), 2.55 (s, 1H), 2.84-3.00 (m, 2H), 3.04-3.16 (m, 2H), 3.39 (sept, J=6.9, 1H), 3.58 (s, 3H), 3.61-3.69 (m, 1H), 3.79-3.84 (m, 4H), 3.98-4.07 (m, 1H), 5.58-5.61 (m, 1H), 5.98-6.02 (m, 1H), 6.67-6.69 (m, 1H), 6.82-6.85 (m, 1H), 6.88-6.91 (m, 1H), 6.99-7.04 (m, 1H), 7.18-7.29 (m, 2H).

LC/MS (Method 2): Rₜ=2.50 min.; MS (ESIpos): m/z=541 [M+H]⁺.

### Example 17

### 8-Chloro-6-(2,3-dimethoxyphenyl)-1-isopropyl-4-(2-morpholin-4-yl-2-oxoethyl)-4H,6H-[2]benzoxepino[4,5-c]isoxazole (Stereoisomer 2)

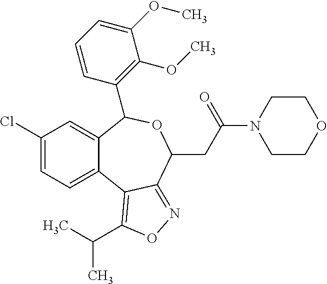

21 mg of stereoisomer 3-2 from Example 3 (0.046 mmol) are dissolved in 3 ml of THF, 31 mg of PyBOP (0.060 mmol) and 8 mg of N,N-diisopropylethylamine (0.060 mmol) are added, and the mixture is stirred at room temperature for 30 min. 5 mg of morpholine (0.060 mmol) are added, and the reaction solution is stirred at room temperature for 16 h. The resulting crude product is purified without further working up directly by preparative HPLC (eluent: acetonitrile/water, gradient 20:80→95:5). 14 mg (58% of theory) of the target compound are obtained.

¹H-NMR (400 MHz, CDCl₃): δ=1.40 (d, J=6.8, 3H), 1.48 (d, J=6.8, 3H), 2.90-3.02 (m, 2H), 3.41-3.50 (m, 3H), 3.55-3.66 (m, 9H), 3.88 (s, 3H), 5.68 (dd, J=8.1, 4.5, 1H), 6.05 (s, 1H), 6.74 (d, J=2.1, 1H), 6.90-6.93 (m, 1H), 6.96-6.99 (m, 1H), 7.08-7.12 (m, 1H), 7.28 (d, J=2.1, 1H), 7.35 (d, J=8.3, 1H).

LC/MS (Method 4): Rₜ=2.80 min.; MS (ESIpos): m/z=527 [M+H]⁺.

### Example 18

### (3R)-1-{[8-Chloro-6-(2,3-dimethoxyphenyl)-1-isopropyl-4H,6H-[2]benzoxepino[4,5-c]isoxazol-4-yl]acetyl}pyrrolidin-3-ol (Stereoisomer 3)

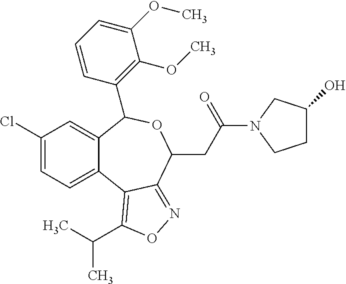

25 mg of stereoisomer 3-3 from Example 3 (0.055 mmol) are dissolved in 1.5 ml of THF, 37 mg of PyBOP (0.071 mmol) and 9 mg of N,N-diisopropylethylamine (0.071 mmol) are added, and the mixture is stirred at room temperature for 30 min. 6 mg of R-3-pyrrolidinol (0.071 mmol) are added, and the reaction solution is stirred at room temperature for 16 h. The resulting crude product is purified without further working up directly by preparative HPLC (eluent: acetonitrile/water, gradient 20:80→95:5). 14 mg (49% of theory) of the target compound are obtained.

LC/MS (Method 4): Rₜ=2.60 min.; MS (ESIpos): m/z=527 [M+H]⁺.

### Example 19

### (3R)-1-{[8-Chloro-6-(2,3-dimethoxyphenyl)-1-isopropyl-4H,6H-[2]benzoxepino[4,5-c]isoxazol-4-yl]acetyl}pyrrolidin-3-ol (Stereoisomer 4)

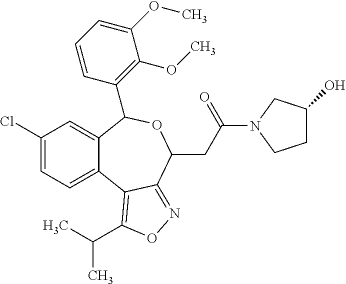

22 mg of stereoisomer 3-4 from Example 3 (0.048 mmol) are dissolved in 1.5 ml of THF, 33 mg of PyBOP (0.062 mmol) and 8 mg of N,N-diisopropylethylamine (0.062 mmol) are added, and the mixture is stirred at room temperature for 30 min. 6 mg of R-3-pyrrolidinol (0.062 mmol) are added, and the reaction solution is stirred at room temperature for 16 h. The resulting crude product is purified without further working up directly by preparative HPLC (eluent: acetonitrile/water, gradient 20:80→95:5). 8 mg (32% of theory) of the target compound are obtained.

¹H-NMR (400 MHz, CDCl₃): δ=1.36 (d, J=6.8, 3H), 1.51 (d, J=6.8, 3H), 1.83-2.11 (m, 2H), 2.95-3.23 (m, 2H), 3.39-3.68 (m, 8H), 3.87 (s, 3H), 4.31-4.47 (m, 1H), 5.29-5.37 (m, 1H), 5.87-5.88 (m, 1H), 6.70-6.72 (m, 1H), 6.92-6.96 (m, 1H), 7.13-7.34 (m, 4H).

LC/MS (Method 3): Rₜ=2.33 min.; MS (ESIpos): m/z=527 [M+H]⁺.

### Example 20

### (3RS)-1-{[8-Chloro-6-(2,3-dimethoxyphenyl)-1-isopropyl-4H,6H-[2]benzoxepino[4,5-c]isoxazol-4-yl]acetyl}pyrrolidin-3-ol (Stereoisomer 4)

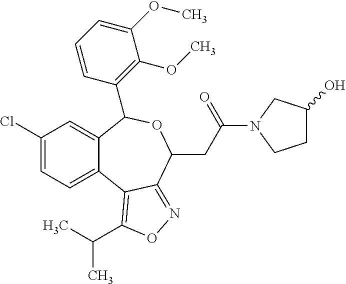

52 mg of stereoisomer 3-4 from Example 3 (0.113 mmol) are dissolved in 4 ml of THF, 76 mg of PyBOP (0.146 mmol) and 19 mg of N,N-diisopropylethylamine (0.146 mmol) are added, and the mixture is stirred at room temperature for 30 min. 13 mg of rac-3-pyrrolidinol (0.146 mmol) are added, and the reaction solution is stirred at room temperature for 16 h. The resulting crude product is purified without further working up directly by preparative HPLC (eluent: acetonitrile/water, gradient 20:80→95:5). 38 mg (62% of theory) of the target compound are obtained.

LC/MS (Method 3): Rₜ=2.32 min.; MS (ESIpos): m/z=527 [M+H]⁺.

### Example 21

### 4-[2-(4-Acetylpiperazin-1-yl)-2-oxoethyl]-8-chloro-6-(2,3-dimethoxyphenyl)-1-isopropyl-4H,6H-[2]benzoxepino[4,5-c]isoxazole (Stereoisomer 2)

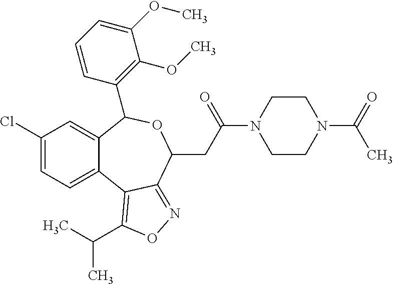

20 mg of stereoisomer 3-2 from Example 3 (0.044 mmol) are dissolved in 3 ml of THF, 30 mg of PyBOP (0.057 mmol) and 7 mg of N,N-diisopropylethylamine (0.057 mmol) are added, and the mixture is stirred at room temperature for 30 min. 7 mg of 1-acetylpiperidine (0.057 mmol) are added, and the reaction solution is stirred at room temperature for 16 h. The resulting crude product is purified without further working up directly by preparative HPLC (eluent: acetonitrile/water, gradient 20:80→95:5). 13 mg (52% of theory) of the target compound are obtained.

LC/MS (Method 4): Rₜ=2.57 min.; MS (ESIpos): m/z=568 [M+H]⁺.

### B. ASSESSMENT OF THE PHARMACOLOGICAL ACTIVITY

The pharmacological effect of the compounds according to the invention can be shown in the following assays:

1. Squalene Synthase Inhibition Assay

a) Obtaining Microsomes:

Microsomes from rat livers are prepared as source of squalene synthase for the activity assay. The rat livers are comminuted and homogenized in twice the volume of homogenization buffer [100 mM Tris/HCl, 0.2 M sucrose, 30 mM nicotinamide, 14 mM sodium fluoride, 5 mM dithiothreitol, 5 mM MgCl₂, protease inhibitor cocktail (from Sigma, Taufkirchen), pH 7.5] (Dounce homogenizer). The supernatant from a 10 000 g centrifugation is then centrifuged at 100 500 g. The pelleted microsomes are taken up in homogenization buffer, diluted to 10 mg/ml protein and stored at −80° C.

b) Squalene Synthase Activity Assay:

The conversion of trans,trans-[1-³H]-famesyl pyrophosphate into [³H]-squalene by the microsomal squalene synthase takes place under the following reaction conditions: rat liver microsomes (protein content 65 μg/ml), 1 mM NADPH, 6 mM glutathione, 10% PBS, 10 mM sodium fluoride, 5 mM MgCl₂, pH 7.5. The compound to be tested in each case is dissolved in DMSO and added to the assay in a defined concentration. The reaction is started by adding famesyl pyrophosphate (final concentration 5 μM) and 20 kBq/ml trans,trans-[1-³H]-famesyl pyrophosphate, and is incubated at 37° C. for 10 min. Subsequently, 100 μl of the reaction solution are mixed with 200 μl of chloroform, 200 μl of methanol and 60 μl of 5 N sodium hydroxide solution and adjusted to 2 mM squalene. After vigorous mixing and subsequent phase separation, an aliquot of the organic phase is transferred into scintillation fluid (Packard Ultima Gold LSC Cocktail) and the organically extractable radioactive compounds are quantified (LS 6500, from Beckman). The reduction in the radioactive signal is directly proportional to the inhibition of squalene synthase by the compound employed in each case.

The exemplary embodiments show IC₅₀ values in the range from 50 nM to 20 μM in this assay.

### 2. Inhibition of Squalene and Cholesterol Synthesis in the Liver of Mice

Male NMR1 mice are kept on a normal rodent diet (NAFAG 3883) in metabolism cages. The light/dark cycle comprises 12 hours, from 06.00 to 18.00 and from 18.00 to 06.00. The animals are employed with a body weight of between 25 g and 40 g in groups of 8-10 animals in the experiments. Feed and drinking water are available to the animals ad libitum.

The substances are, according to their solubility, administered orally in aqueous tragacanth suspension (0.5%) or in Solutol HS15/saline solution (20:80) by gavage in a volume of 10 ml/kg of body weight or else injected subcutaneously in Solutol HS15/saline solution (20:80) or DMSO/saline solution (20:80). The corresponding control groups receive only the corresponding formulating agent without active substance. One or two hours after administration of the substance, the animals receive intraperitoneal injections of radiolabeled ¹⁴C-mevalonolactone. One or two hours after the ¹⁴C-mevalonolactone injection, or 2-4 hours after the administration of substance, the animals are sacrificed, the abdominal cavity is opened, and liver tissue is removed. Immediately after removal, the tissue is dried on the surface, weighed and homogenized in isopropanol. The further processing and extraction of the synthesized squalene and its secondary products takes place by a method of I. Duncan et al. (J. Chromatogr. 1979, 162), modified by H. Bischoff et al. (Atherosclerosis 1997, 135).

The extracted lipid fraction is taken up in 1 ml of isopropanol, transferred into scintillation vials, made up with 15 ml of Ultima Gold® scintillation fluid (Packard) and counted in a liquid scintillation counter (Beckmann Coulter LS 6500).

After calculation of the specific ¹⁴C activity of the lipid fraction (dpm/g of liver tissue), the rate of synthesis of the radiolabeled ¹⁴C squalene and of the ¹⁴C secondary metabolites of the animals treated with the active substance is compared with the rate of synthesis of the radiolabelled ¹⁴C squalene and of the ¹⁴C secondary metabolites of the control animals treated only with formulating agent. A reduction in the rate of synthesis by ≧30% compared with the rate of synthesis for the control animals (=100%) is regarded as pharmacologically active if the statistical assessment by Student's t test results in a p value of <0.05.

### 3. Inhibition of Squalene and Cholesterol Synthesis in the Liver of Rats

Male Wistar rats are kept on a normal rodent diet (NAFAG 3883) in Makrolon® type III cages. The light/dark cycle comprises 12 hours, from 06.00 to 18.00 and from 18.00 to 06.00. The animals are employed with a body weight of between 150 g and 200 g in groups of 6-8 animals in the experiments. The feed is withdrawn from the animals 18-22 hours before the start of the experiment; drinking water is available ad libitum up to the end of the experiment.

The substances are, according to their solubility, administered orally in aqueous tragacanth suspension (0.5%) or in Solutol HS15/saline solution (20:80) by gavage in a volume of 10 ml/kg of body weight or else injected subcutaneously in Solutol HS15/saline solution (20:80) or DMSO/saline solution (20:80). The corresponding control groups receive only the corresponding formulating agent without active substance. One or two hours after administration of the substance, the animals receive intraperitoneal injections of radiolabelled ¹⁴C-mevalonolactone. One or two hours after the ¹⁴C-mevalonolactone injection, or 2-4 hours after the administration of substance, the animals are sacrificed, the abdominal cavity is opened, and liver tissue is removed. Immediately after removal, the tissue is dried on the surface, weighed and homogenized in isopropanol. The further processing and extraction of the synthesized squalene and its secondary products takes place by a method of I. Duncan et al. (J. Chromatogr. 1979, 162), modified by H. Bischoff et al. (Atherosclerosis 1997, 135).

The extracted lipid fraction is taken up in 1 ml of isopropanol, transferred into scintillation vials, made up with 15 ml of Ultima Gold scintillation fluid (Packard) and counted in a liquid scintillation counter (Beckmann Coulter LS 6500).

After calculation of the specific ¹⁴C activity of the lipid fraction (dpm/g of liver tissue), the rate of synthesis of the radiolabelled ¹⁴C squalene and of the ¹⁴C secondary metabolites of the animals treated with the active substance is compared with the rate of synthesis of the radiolabelled ¹⁴C squalene and of the ¹⁴C secondary metabolites of the control animals treated only with formulating agent. A reduction in the rate of synthesis by ≧30% compared with the rate of synthesis for the control animals (=100%) is regarded as phannacologically active if the statistical assessment by Student's t test results in a p value of <0.05.

### C. EXEMPLARY EMBODIMENTS OF PHARMACEUTICAL COMPOSITIONS

The compounds according to the invention can be converted into pharmaceutical preparations in the following ways:

Tablet:

Composition:

100 mg of the compound according to the invention, 50 mg of lactose (monohydrate), 50 mg of maize starch (native), 10 mg of polyvinylpyrrolidone (PVP 25) (from BASF, Ludwigshafen, Germany) and 2 mg of magnesium stearate.

Tablet weight 212 mg, diameter 8 mm, radius of curvature 12 mm.

Production:

A mixture of compound according to the invention, lactose and starch is granulated with a 5% strength solution (m/m) of the PVP in water. The granules are dried and mixed with the magnesium stearate for 5 minutes. This mixture is compressed in a conventional tablet press (see above for format of the tablet). A guideline compressive force for the compression is 15 kN.

Suspension which can be Administered Orally:

Composition:

1000 mg of the compound according to the invention, 1000 mg of ethanol (96%), 400 mg of Rhodigel® (xanthan gum from FMC, Pennsylvania, USA) and 99 g of water.

10 ml of oral suspension correspond to a single dose of 100 mg of the compound according to the invention.

Production:

The Rhodigel is suspended in ethanol, and the compound according to the invention is added to the suspension. The water is added while stirring. The mixture is stirred for about 6 h until the swelling of the Rhodigel is complete.

Solution which can be Administered Orally:

Composition:

500 mg of the compound according to the invention, 2.5 g of polysorbate and 97 g of polyethylene glycol 400. 20 g of oral solution correspond to a single dose of 100 mg of the compound according to the invention.

Production:

The compound according to the invention is suspended in the mixture of polyethylene glycol and polysorbate with stirring. The stirring process is continued until the compound according to the invention has completely dissolved.

i.v. Solution:

The compound according to the invention is dissolved in a concentration below the saturation solubility in a physiologically tolerated solvent (e.g. isotonic saline, 5% glucose solution and/or 30% PEG 400 solution). The solution is sterilized by filtration and used to fill sterile and pyrogen-free injection containers.

1. A compound of the formula (I) 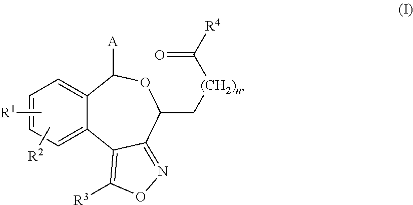
in which
A is (C₆-C₁₀)-aryl or 5- to 10-membered heteroaryl, which may each be substituted up to three times, identically or differently, by substituents selected from the group of halogen, cyano, nitro, trifluoromethyl, trifluoromethoxy, (C₁-C₆)-alkyl, (C₂-C₆)-alkenyl, (C₂-C₆)-alkynyl, (C₁-C₆)-alkoxy, hydroxy, amino, mono- and di-(C₁-C₆)-alkylamino,
or
is a group of the formula 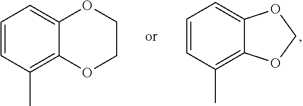
n is the number 0, 1, 2 or 3,
R¹ and R² are identical or different and are independently of one another, hydrogen, halogen, cyano, nitro, trifluoromethyl, trifluoromethoxy, (C₁-C₆)-alkyl or (C₁-C₆)-alkoxy,
R³ is (C₁-C₈)-alkyl, (C₂-C₈)-alkenyl, (C₂-C₈)-alkynyl, each of which may be substituted by (C₃-C₈)-cycloalkyl, or is (C₃-C₈)-cycloalkyl, where (C₁-C₈)-alkyl, (C₂-C₈)-alkenyl, (C₂-C₈)-alkynyl and (C₃-C₈)-cycloalkyl may each be substituted by fluorine, hydroxy, amino, (C₁-C₄)-alkoxy or (C₁-C₄)-acyloxy,
and
R⁴ is a group of the formula —OR⁵ or —NR⁶R⁷ in which
R⁵ is hydrogen or (C₁-C₆)-alkyl,
R⁶ and R⁷ are identical or different and are independently of one another hydrogen, (C₁-C₆)-alkyl or (C₃-C₈)-cycloalkyl, each of which may be substituted by substituents selected from the group of carboxyl, (C₁-C₆)-alkoxycarbonyl, aminocarbonyl, mono- and di-(C₁-C₆)-alkylaminocarbonyl,
or
R⁶ and R⁷ together with the nitrogen atom to which they are bonded form a 4- to 8-membered heterocycle which may comprise a further ring heteroatom from the series N—R⁸, O, S, SO or SO₂ and be substituted by substituents selected from the group of hydroxy, oxo, amino, (C₁-C₆)-alkyl, carboxyl, (C₁-C₆)-alkoxycarbonyl, aminocarbonyl, mono- and di-(C₁-C₆)-alkylaminocarbonyl, in which
(C₁-C₆)-alkyl may in turn be substituted by substituents selected from the group of fluorine, hydroxy, amino, carboxyl, (C₁-C₆)-alkoxycarbonyl, aminocarbonyl, mono- and di-(C₁-C₆)-alkylaminocarbonyl,
and
R⁸ is hydrogen, (C₁-C₄)-alkyl, (C₁-C₄)-acyl or (C₁-C₄)-alkoxycarbonyl,
and the salts, solvates and solvates of the salts thereof.

2. A compound of the formula (I) as claimed in claim 1, in which
A is phenyl, naphthyl or pyridyl, each of which may be substituted up to twice, identically or differently, by substituents selected from the group of fluorine, chlorine, bromine, cyano, nitro, trifluoromethyl, trifluoromethoxy, (C₁-C₄)-alkyl, (C₁-C₄)-alkoxy, hydroxy, amino, mono- and di-(C₁-C₄)-alkylamino,
n is the number 0 or 1,
R¹ and R² are identical or different and are independently of one another, hydrogen, fluorine, chlorine, bromine, cyano, nitro, trifluoromethyl, trifluoromethoxy, (C₁-C₄)-alkyl or (C₁-C₄)-alkoxy,
R³ is (C₁-C₆)-alkyl which may be substituted by (C₃-C₆)-cycloalkyl, or is (C₃-C₆)-cycloalkyl, where
(C₁-C₆)-alkyl and (C₃-C₆)-cycloalkyl may each be substituted by hydroxy, amino, (C₁-C₄)-alkoxy or (C₁-C₄)-acyloxy,
and
R⁴ is a group of the formula —OR⁵ or —NR⁶R⁷ in which
R⁵ is hydrogen or (C₁-C₆)-alkyl,
R⁶ and R⁷ are identical or different and are independently of one another hydrogen, (C₁-C₆)-alkyl or (C₃-C₆)-cycloalkyl, each of which may be substituted by substituents selected from the group of carboxyl, (C₁-C₆)-alkoxycarbonyl, aminocarbonyl, mono- and di-(C₁-C₆)-alkylaminocarbonyl,
or
R⁶ and R⁷ together with the nitrogen atom to which they are bonded form a 5- to 7-membered heterocycle which may comprise a further ring heteroatom from the series N—R⁸, O or S and be substituted by substituents selected from the group of hydroxy, oxo, amino, (C₁-C₆)-alkyl, carboxyl, (C₁-C₆)-alkoxycarbonyl, aminocarbonyl, mono- and di-(C₁-C₆)-alkylaminocarbonyl, in which
(C₁-C₆)-alkyl in turn may be substituted by substituents selected from the group of hydroxy, amino, carboxyl, (C₁-C₆)-alkoxycarbonyl, aminocarbonyl, mono- and di-(C₁-C₆)-alkylaminocarbonyl,
and
R⁸ is hydrogen, (C₁-C₄)-alkyl, (C₁-C₄)-acyl or (C₁-C₄)-alkoxycarbonyl,
and the salts, solvates and solvates of the salts thereof.

3. A compound of the formula (I) as claimed in claim 1, in which
A is phenyl which is substituted once or twice, identically or differently, by fluorine, chlorine, bromine, methyl, methoxy, ethoxy or dimethylamino,
n is the number 0,
R¹ and R² are independently of one another hydrogen or chlorine,
R³ is (C₁-C₆)-alkyl which may be substituted by (C₃-C₆)-cycloalkyl, or is (C₃-C₆)-cycloalkyl, where
(C₁-C₆)-alkyl and (C₃-C₆)-cycloalkyl may each be substituted by hydroxy or (C₁-C₄)-acyloxy,
and
R⁴ is a group of the formula —OR⁵ or —NR⁶R⁷ in which
R⁵ is hydrogen or (C₁-C₄)-alkyl,
R⁶ and R⁷ are identical or different and are independently of one another hydrogen or (C₁-C₄)-alkyl which may be substituted by carboxyl or (C₁-C₄)-alkoxycarbonyl,
or
R⁶ and R⁷ together with the nitrogen atom to which they are bonded form a 5- or 6-membered heterocycle which may comprise a further ring heteroatom from the series N—R⁸ and O and be substituted by substituents selected from the group of hydroxy, oxo, amino, (C₁-C₄)-alkyl, carboxyl, (C₁-C₄)-alkoxycarbonyl, aminocarbonyl, mono- and di-(C₁-C₄)-alkylaminocarbonyl, in which
(C₁-C₄)-alkyl in turn may be substituted by substituents selected from the group of hydroxy, amino, carboxyl, (C₁-C₄)-alkoxycarbonyl, aminocarbonyl, mono- and di-(C₁-C₄)-alkylaminocarbonyl,
and
R⁸ is hydrogen, (C₁-C₄)-alkyl or (C₁-C₄)-acyl,
and the salts, solvates and solvates of the salts thereof.

4. A compound of the formula (I) as claimed in claim 1, in which
A is phenyl which is substituted once or twice, identically or differently, by fluorine, chlorine, methyl, methoxy or ethoxy,
n is the number 0,
R¹ and R² are independently of one another hydrogen or chlorine,
R³ is (C₁-C₆)-alkyl,
and
R⁴ is hydroxy or a group of the formula —NR⁶R⁷ in which
R⁶ and R⁷ together with the nitrogen atom to which they are bonded form a 5- or 6-membered heterocycle which may comprise a further ring heteroatom from the series N—R⁸ and O and be substituted by hydroxy, oxo, (C₁-C₄)-alkyl, carboxyl or (C₁-C₄)-alkoxycarbonyl, in which
(C₁-C₄)-alkyl in turn may be substituted by hydroxy, carboxyl or (C₁-C₄)-alkoxycarbonyl,
and
R⁸ is hydrogen, methyl or acetyl,
and the salts, solvates and solvates of the salts thereof.

5. A process for preparing a compound of the formula (I) as defined in claim 1, characterized in that a compound of the formula (VIII) 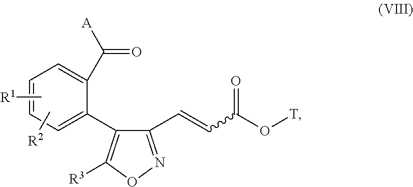
in which R¹, R², R³ and A each have the meanings indicated in claim to 4, and T is (C₁-C₄)-alkyl,
is reduced in an inert solvent with the aid of a boron hydride or aluminum hydride to a compound of the formula (IX) 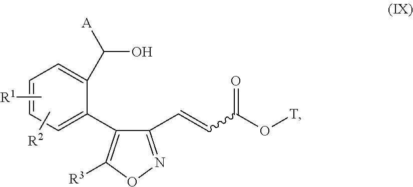
in which R¹, R², R³, A and T each have the meanings indicated above,
the latter is subsequently cyclized in an inert solvent in the presence of a base to give a compound of the formula (I-A) 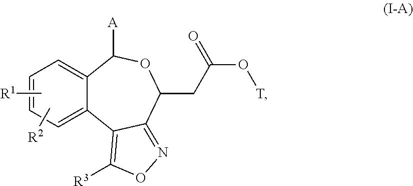
in which R¹, R², R³, A and T each have the meanings indicated above,
then hydrolyzed under acidic conditions to give a carboxylic acid of the formula (I-B) 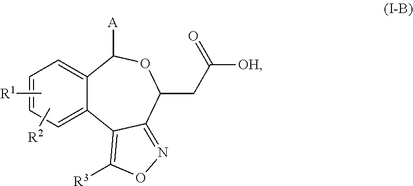
in which R¹, R², R³ and A each have the meanings indicated above,
the compounds of the formula (I-B) are where appropriate converted by methods known from the literature for chain extension into the homologous carboxylic acids of the formula (I-C) 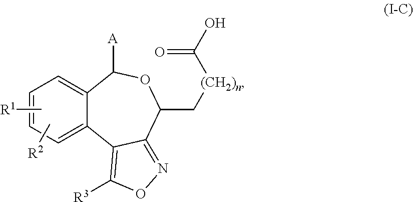
in which R¹, R², R³, A and n each have the meanings indicated in claim 1, but where n is not the number 0,
and the resulting compounds of the formula (I-B) or (I-C) are then converted by methods known from the literature for esterification or amidation of carboxylic acids with a compound of the formula (X) or (XI) 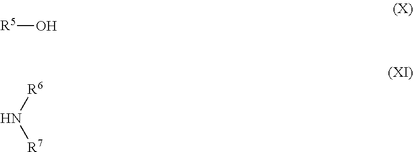
in which R⁵, R⁶ and R⁷ each have the meanings indicated in claim 1, but
where R⁵ is not hydrogen,
into the compounds of the formula (I),
and the compounds of the formula (I) are where appropriate separated into the stereochemically pure isomers and/or converted with the appropriate (i) solvents and/or (ii) bases or acids into the solvates, salts and/or solvates of the salts thereof.

6. A compound of the formula (I) as defined in claim 1 for the treatment and/or prophylaxis of diseases.

7. (canceled)

8. A pharmaceutical composition comprising a compound of the formula (I) as defined in claim 4 in combination with one or more further active ingredients selected from the group consisting of cholesterol-lowering statins, cholesterol absorption inhibitors, HDL-elevating, triglyceride-lowering and/or apolipoprotein B-lowering substances, oxidation inhibitors and compounds having antiinflammatory activity.

9. A pharmaceutical composition comprising a compound of the formula (I) as defined in claim 1 in combination with an inert, non-toxic, pharmaceutically suitable excipient.

10. The pharmaceutical composition as claimed in claim 8 for the treatment and/or prophylaxis of dyslipidemias, arteriosclerosis, restenosis and ischemias.

11. A method for the treatment and/or prophylaxis by using of dyslipidemias, arteriosclerosis, restenosis and ischemias in humans and animals by using an effective amount of at least one compound of the formula (I) as defined in claim 1.
